(19) United States
(12) Patent Application Publication
(10) Pub. No.: US 2022/0054007 A1
(43) Pub. Date: Feb. 24, 2022

(54) IMPLANTABLE INTRAOCULAR PRESSURE SENSORS AND CALIBRATION

(71) Applicant: Qura, Inc., Framingham, MA (US)

(72) Inventors: Douglas P. Adams, Sudbury, MA (US); Amitava Gupta, Roanoke, VA (US)

(73) Assignee: Qura, Inc., Framingham, MA (US)

(21) Appl. No.: 17/389,408

(22) Filed: Jul. 30, 2021

Related U.S. Application Data
(63) Continuation of application No. PCT/US2020/015869, filed on Jan. 30, 2020.
(60) Provisional application No. 62/798,919, filed on Jan. 30, 2019.

(51) Int. Cl. A61B 3/16; A61B 5/00; G16H 40/40

(52) U.S. Cl. CPC A61B 3/16; A61B 5/721; A61B 5/1103; A61B 2560/0223; A61B 2560/0219; G16H 40/40

(57) ABSTRACT

Intraocular pressure sensing devices and methods of use. The intraocular pressure sensing devices may include one or more calibration sensors that are adapted to sense fibrotic growth over the implant post-implantation. Methods can take into account the amount of fibrosis over the implant, and its effect on IOP, when calculating the subject's IOP. Additionally, methods herein can calculate IOP while factoring in blink-induced variation in IOP.

### CROSS REFERENCE TO RELATED APPLICATIONS

This application is a bypass continuation of International Application No. PCT/US2020/015869, filed Jan. 30, 2020, which in turn claims priority to U.S. Provisional Application No. 62/798,919, filed Jan. 30, 2019. Each of these applications is incorporated by reference herein for all purposes.

This disclosure is related to PCT Pubs. WO2017/210316, WO2019/191748, WO/2019/164940, and incorporates by reference herein the entire disclosures thereof for all purposes.

### INCORPORATION BY REFERENCE

All publications and patent applications mentioned in this specification are herein incorporated by reference to the same extent as if each individual publication or patent application was specifically and individually indicated to be incorporated by reference.

### BACKGROUND

Glaucoma is second only to cataract as a leading cause of global blindness and is the leading cause of irreversible visual loss. Worldwide, there were 60.5 million people with open angle glaucoma and angle closure glaucoma in 2010, projected to increase to 79.6 million by 2020, and of these, 74% will have OAG. (Quigley and Broman, in Br J Ophthalmol. 2006; 90(3), pp 262-267). Bilateral blindness from glaucoma is projected to affect greater than 11 million by 2020 globally. Risk factors for open-angle glaucoma include increased age, African ethnicity, family history, increased intraocular pressure, myopia, and decreased corneal thickness. Risk factors for angle closure glaucoma include Inuit and Asian ethnicity, hyperopia, female sex, shallow anterior chamber, short axial length, small corneal diameter, steep corneal curvature, shallow limbal chamber depth, and thick, relatively anteriorly positioned intraocular lens.

Elevated intraocular pressure (“IOP”) is the most important known risk factor for the development of POAG, and its reduction remains the only clearly proven treatment. Several studies have confirmed that reduction of IOP at any point along the spectrum of disease severity reduces progression (Early Manifest Glaucoma Treatment Trial to Advanced Glaucoma Intervention Study). Also, IOP reduction reduces the development of POAG in patients with ocular hypertension (OHT) and reduces progression in patients with glaucoma despite normal IOP, as seen in the Collaborative Normal Tension Glaucoma Study. The normal IOP for 95% of Caucasians is within the range of 10-21 mm Hg. The EGPS and Early Manifest Glaucoma Treatment Trial found that long-term IOP fluctuations were not associated with progression of glaucoma, while the AGIS study found an increased risk of glaucoma progression with increased long-term IOP fluctuation, especially in patients with low IOP.

Current monitoring of IOP occurs in the offices of a vision care practitioner, typically an ophthalmologist, ranging from once a year to once every 3-6 months, once glaucoma is diagnosed. It is known that IOP varies over a wide range in individuals, including a diurnal fluctuation, longer term variations and occurrence of spikes in IOP, therefore a single measurement cannot provide adequate data to diagnose an elevated IOP, requiring prescription of pressure regulating or pressure reducing medication. Treatment options for reduction of IOP include medical therapy, such as beta blockers, alpha agonists, miotics, carbonic anhydrase inhibitors, and prostaglandin analogues, administered as eyedrops, up to 4 times a day; laser treatment, such as argon laser trabeculoplasty (ALT), selective laser trabeculoplasty (SLT), neodymium-doped yttrium aluminum garnet (Nd:YAG) laser iridotomy, diode laser cycloablation, and laser iridoplasty; surgical procedures including iris procedures (e.g., peripheral iridectomy), angle procedures (e.g., goniotomy and trabeculotomy), filtration procedures (e.g., trabeculectomy) and non-penetrating filtration procedures (e.g., deep sclerectomy and viscocanalostomy); and drainage shunts including episcleral implants (e.g., Molteno, Baerveldt, and Ahmed) or mini-shunts (e.g., ExPress Mini Shunt and iStent).

A substantial majority of glaucoma patients are treated by medication to control IOP, sometimes over three decades. Patients treated surgically or using laser treatment may also be administered medication. Lack of compliance of patients to long term medication protocols is exacerbated by advancing age and lack of positive concrete immediate incentives.

Continuous monitoring of IOP replaces the standard practice of monitoring IOP episodically, and hence provides a more accurate and detailed account of patient compliance, enabling the caregiver to take steps to take additional steps to enhance compliance if required.

Monitoring efficacy of prescribed treatment via continuous IOP data following a change in treatment modality or protocol provides the caregiver with a prompt feedback on the efficacy of the change in treatment and thereby supports a better outcome.

Post market monitoring of approved glaucoma treatments, especially newly approved glaucoma treatments may require post market monitoring by health care agencies in order to monitor safety and efficacy on the targeted patient population. Data from continuous monitoring of IOP may be submitted by manufacturers of newly approved drugs or devices to meet this requirement.

Data recorded may be used by clinical researchers to monitor efficacy and may be submitted to regulatory authorities for prompt approval, if the results so warrant.

The references immediately below describe some previous concepts related to monitoring intraocular pressure.

1. “An implantable microfluidic device for self-monitoring of intraocular pressure”, by Mandel, Quake, Su and Araci, in Nature Medicine 20, 1074-1078 (2014), in which three images of a microfluidic intraocular sensor are shown.

2. “Implantable parylene-based wireless intraocular pressure sensor”, by Chen, Rodger, Saati, Humayun and Tai in IEEE 21^{st} International Conference on Micro Electro Mechanical Systems, 2008. MEMS 2008. This paper presents an implantable, wireless, passive pressure sensor for ophthalmic applications.

3. “Rollable and implantable intraocular pressure sensor for the continuous adaptive management of glaucoma”, Piffaretti, Barrettino, Orsatti, Leoni, Stegmaier, in Conference Proceedings IEEE Eng Med Biol Soc, 2013; 2013:3198-201. doi: 10.1109/EMBC.2013.6610221.

4. “Implantable microsensor, telemetrically powered and read out by patient hand-held device”, by Implandata Ophthalmic Products GmbH Kokenstrasse 5 30159 Hannover Germany, 2014. The Eyemate® by Implandata Ophthalmic Products GmbH is an additional example. IOP data reported on human patients show a substantial and unexplained drop, possibly indicating loss of sensor sensitivity upon deposition of fibrous tissue.

5. “Preliminary study on implantable inductive-type sensor for continuous monitoring of intraocular pressure”, by Kim Y W, Kim M J, Park, Jeoung, Kim S H, Jang, Lee, Kim J H, Lee, and Kang in Clinical & Experimental Ophthalmology, 43(9), pp 830-837, 2015.

6. “An intra-ocular pressure sensor based on a glass reflow process”, by Haque and Wise in Solid-State Sensors, Actuators, and Microsystems Workshop, Hilton Head Island, S.C., Jun. 6-10, 2010.

7. Some earlier approaches used a capacitive-based membrane pressure sensor. For example, a diaphragm can deflect under pressure, changing the effective distance between two parallel plates, and thus increasing the measured capacitance across the plates. An example is “Miniaturized implantable pressure and oxygen sensors based on polydimethylsiloxane thin films”, Koley, Liu, Nomani, Yim, Wen, Hsia: in Mater. Sci. Eng. C 2009, 29, 685-690.

8. “Microfabricated implantable Parylene-based wireless passive intraocular pressure sensors”, by Chen, Rodger, Saati, Humayun, Tai: J. Microelectromech. Syst. 2008, 17, 1342-1351.

9. “An Implantable, All-Optical Sensor for Intraocular Pressure Monitoring”, by Hastings, Deokule, Britt and Brockman in Investigative Ophthalmology & Visual Science, 2012. Vol. 53, pp 5039, in which an approach to IOP monitoring based on a near infrared (NIR) image of an implanted micromechanical sensor is presented.

10. “Implant Device, Sensor Module, Single Use Injector and Method for Producing an Implant Device”, U.S. Pat. No. 9,468,522 B2, by Sholten, D., October, 2016, which does not address the durability and continued functionality of the sensor post-implantation, even though continued function of the pressure sensor is a critical requirement for efficacy of the device.

11. “Chronically Implanted Pressure Sensors: Challenges and State of the Field”, A Review by Yu, Kim and Meng, in Sensors 2014, 14, 20620-20644; doi:10.3390/s141120620.

12. “Polymer-based miniature flexible capacitive pressure sensor for intraocular pressure (IOP) monitoring inside a mouse eye”, by Ha, de Vries, John, Irazoqui, and Chappell in Biomed Microdevices (2012) 14:207-215, DOI 10.1007/s10544-011-9598-3.

13. “Pressure Sensors for Small scale Applications and Related Methods”, U.S. Pat. No. 9,596,988 B2, by Irazoqui, Ha, Chappelle, and John, 2017, which describes substantial deposits of fibrous material on the implanted sensor in animal models within a relatively short period (7-41 days) after implantation for all encapsulation designs that they tested (FIGS. 39, 40 and 41).

14. “Implantation and testing of a novel episcleral pressure transducer: A new approach to telemetric intraocular pressure monitoring”, by Mariacher, Ebner, et al, in Experimental Eye Research, (2018) 166, 84-90. In this recent report on in-vivo performance of an implanted IOP sensor, the authors report that every measurement required a calibration, presumably because ocular environment in rabbit models caused a change in the response of the sensor to pressure variations.

15. Yu, L., Kim, B. J., and Meng, E., “Chronically Implanted Pressure Sensors: Challenges and State of the Field”, in Sensors (2014), 14, 20620-20644; doi:10.3390/s141120620. In this review, the authors address the issue of immune response or biofouling subsequent to implantation that affect sensor performance.

16. Coleman, J, and Trokel, S, “Direct-Recorded Intraocular Pressure Variations in a Human Subject”, in Arch Ophthalmol, 1989, 82, 637-640.

17. Downs J. C., Burgoyne C. F., et al, “24-hour IOP telemetry in the nonhuman primate: implant system performance and initial characterization of IOP at multiple timescales”, Invest Ophthalmol Vis Sci. 2011; 52(10): 7365-7375.

18. Tsubota, K., “Tear Dynamics and Dry Eye” in Progress in Retinal and Eye Research, 1998, 17, 4, 565.

Any change in the response of the implanted sensor (either the slope or the intercept of the plot of measured pressure), calculated from the current output using a calibration curve supplied with each sensor vs. reference pressure (e.g. FIG. 22) requires the sensor to be recalibrated at the time measurement is taken. Nominally, the implanted sensor can also be calibrated by normalizing IOP data provided by the sensor to IOP data obtained by tonometry at the doctor's office. Unless eliminated, need of such calibration renders the implant unusable for at home measurements, since calibration cannot be performed by test subjects or human patients. There is an unmet need to develop intraocular pressure sensors that are not affected by prolonged exposure to the ocular environment, such that their output can be used to reliably calculate and monitor intraocular pressure.

### SUMMARY OF THE DISCLOSURE

The present disclosure relates to intraocular pressure sensors and methods of calibrating the output from the pressure sensors. In some embodiments, the calibration takes into account tissue growth on the implantable device, which can influence the pressure sensor output. By taking into account tissue growth on the implant (including the pressure sensor), the output from the pressure sensor can be appropriately modified to take into account the tissue growth, and thus the system and methods can determine an accurate intraocular pressure. Without taking tissue growth on the implant into consideration, the output from the pressure sensor may not be accurate, due to tissue that has grown over the pressure sensor and changed the pressure sensor sensitivity to changes in ambient pressure.

One aspect of the disclosure is a hermetically sealed implantable intraocular pressure sensor assembly adapted to wirelessly communicate with an external device. The assembly can include a hermetically sealed housing, the hermetically sealed housing can include therein: an antenna in electrical communication with a rechargeable power source, the rechargeable power source in electrical communication with an ASIC, and the ASIC in electrical communication with a pressure sensor. An exemplary intraocular pressure sensing implant is shown in FIG. 26.

In some embodiments, an ASIC in the implant is also connected to a second sensor positioned adjacent to the pressure sensor assembly, such that the second sensor is adapted to monitor the mass of fibrous tissue deposited on the surface of the hermetic seal. This sensor is considered a calibration sensor, and may be a mass sensor which can be, without limitation, a quartz microbalance, a surface acoustic wave sensor, or any other type of sensor that monitors the magnitude of the mass of deposits that collect on the surface of the hermetically sealing surface, or the surface of an additional biocompatible coating that may be applied in order to minimize post-operative inflammation. Any of the implantable pressures herein can thus include a housing that comprises a pressure sensor and a calibration sensor.

In some embodiments, including any of the claims herein, the sensitivity of the mass sensor may be better than 1 picogram of deposit per cm² of implant surface. A mass sensor can be calibrated during assembly, and again just prior to implantation while the implant is enclosed in a sterile package. Calibration of the mass sensor can include measurement of its electric response as a function of controlled magnitudes of deposits added to the surface of the implant, at multiple pressure environments. The reading of the mass sensor is monitored and recorded at the same time as the reading of the pressure sensor, and the two readings can thus be correlated. The calibration sensor can thus be used to perform an in-situ calibration of the reading of the pressure sensor whenever IOP data is collected from the intraocular pressure sensor. Thus, even if an intraocular pressure sensing implant undergoes tissue growth thereon post-implantation due to a fibrotic response, the system can take the tissue growth into consideration and modify the pressure sensor output based on the amount of tissue growth.

In some embodiments, an ASIC in the implant comprises a signal processing mechanism or means that comprises an electronic band pass filter, spectral analysis using a fast Fourier transform, or a Kalman filter designed to measure the mean transient increase in IOP due to a blink, occurring over 100-500 msec, in some preferred embodiments over 150-350 msec. Natural blinks cause a transient increase in IOP lasting for 100-500 milliseconds, preferably 150-350 msec. An average person blinks at the rate of 10-30 blinks per minute, average 14+/−4 blinks/minute. Blink rate changes with visual behavior, for example, reading or prolonged visual engagement with a video screen slows down blink rate. Blink rate are also affected by ocular disorders, especially corneal surface disorders, such as dry eye. This transient increase in IOP is species and patient specific, and depends on the biomechanics of the sclera as well as the blink forced applied by the eyelids on the cornea. This disclosure describes how a transient increase in IOP can be used as part of a calibration process, especially during the period between routine eye exams that are generally conducted every 6 months on healthy, non-glaucomatous patients. Alternatively, such signal processing may be performed in an external unit which receives the IOP outputs wirelessly from the implant.

In some embodiments, the antenna is part of a first circuit adapted to supply power to the rechargeable power source and also part of a second circuit adapted to transmit data to the external device.

In some embodiments, the assembly further comprises a flexible circuit, the flexible circuit in electrical communication with the pressure sensor and the ASIC. The flexible circuit can be in electrical communication with the antenna and the power source.

In some embodiments, the assembly further comprises a multilayer coating comprised of alternate layers of Paralyne C and SiOx. Each layer may have a thickness of 0.1-1.0 microns, and up to 20 layers may be applied through a vacuum deposition process, such as chemical vapor deposition.

In some embodiments, the multilayer coating may be further coated with a hydrogel coating comprised of a hydrophilic or amphiphilic cross-linked polymer, wherein said hydrogel layer has a gradient in cross-link density. The hydrogel layer can have a gradient in number density of hydroxyl groups, said gradient being in the opposite direction of the gradient in cross-link density. The hydrogel layer can be impregnated with an anticlotting agent. The hydrogel layer can be impregnated with an anti-inflammatory agent. An outer surface of the hydrogel coating can be textured to stimulate a controlled fibrotic response. The coating can be infused with at least one of an anti-inflammatory agent and an anticlotting agent. The coating can be chemically bonded to medicaments that are slowly and sustainably released into the eye over a period of not less than 10 days. The textured surface can include a plurality of depressions, each of which have a height between 5 microns and 15 microns, such as 7.5 microns and 12.5 microns, such as 10 microns.

In some embodiments, the pressure sensor comprises a hermetically sealed module comprising an inert fluid situated inside the module. The hermetic seal encasing said pressure sensor can include a Titanium foil of thickness in the range of 5-25 microns, the foil being undulated to enhance its surface area and resistance to mechanical stress.

In some embodiments, the sensor can comprise a piezoelectric sensing element wherein said inert fluid of claim 12 transmits hydrostatic pressure to said sensing element through said Titanium foil. The sensor can comprise a capacitative sensing element wherein said inert fluid of claim 12 transmits hydrostatic pressure to said sensing element. The sensor can have dimensions of length 0.2 mm to 1.5 mm in length, 0.2 mm to 0.7 mm in width and 0.1 mm to 0.7 mm in thickness.

In some embodiments, the antenna has a space filling design, wherein the antenna is connected to an electrical circuit that can be adjusted for its electrical impedance as a function of its resistive load. The antenna can be disposed on a ceramic substrate situated inside a Titanium casing, wherein said antenna assembly being of thickness in the range 100-500 microns. The circuit comprising the antenna can have a Q factor in the range of 10-50 under use conditions. The antenna can be comprised of vacuum deposited metal filaments on a ceramic substrate. The antenna can provide both data transfer and energy transfer functions. The antenna can comprise a conductive length of no less than 15 mm and no more than 100 mm. The antenna can transmit electromagnetic energy at a frequency that is not harmful to the human body.

In some embodiments, the ASIC comprises a microelectronic circuit comprising a microcontroller, a flash memory, a non-volatile memory and a logic circuit. The logic circuit can comprise power management and data management modules. The ASIC comprises a microelectronic circuit wherein said microelectronic circuit comprises conductive connectors of width in the range 36-360 nanometers.

In some embodiments, the ASIC is positioned on the same silicon wafer as the pressure sensor and the mass sensor, thus reducing form factor.

In some embodiments, the implantable assembly has a length not greater than 4.8 mm (e.g., not greater than 4.5 mm), a height not greater than 1.5 mm, and a width not greater than 1.5 mm.

In some embodiments, the pressure sensor is on die, in other words, positioned on the same silicon wafer that comprises the ASIC.

In some embodiments, the pressure sensor element is covered with a fluid or a non-compressible, biocompatible gel such as silastic, a medical grade adhesive manufactured by Du Pont Corporation or siluron, a silicone oil manufactured by Fluoron. The fluid or gel is then covered or coated with a flexible coating adapted to transmit pressure from the external environment to a fluid within the fluid filled chamber. The flexible coating can be a multilayer coating, of overall thickness 5-20 microns, such as 7-17 microns. The multilayer coating can comprise alternate layers of Paralyne C and SiOx.

In some embodiments, the pressure sensor is adapted to sense intraocular pressure more than once every 12 hours and no more than once every 10 milliseconds, and wherein the ASIC is adapted to facilitate the storage of pressure data more than once every 12 hours and no more than once every 10 milliseconds.

In some embodiments, the assembly further comprises an external device in wireless communication with the implantable assembly. The external device can have a communication component that is adapted to transmit a wireless signal to the implantable assembly indicating its readiness to receive data from the implantable assembly and provide wireless power to the implantable assembly, and wherein the ASIC is adapted to acknowledge the transmitted wireless signal with one of at least two different signals, indicating its readiness to transmit or receive data and its readiness to receive wireless power.

In some embodiments, the ASIC has a communication component that is adapted to transmit pressure data from the implantable assembly to the external device, wherein the external device has a communication component that is adapted to receive the transmitted pressure data, wherein the ASIC is adapted to transmit the pressure data upon receiving a trigger signal from the external device and after acknowledging the receipt of the trigger signal.

In some embodiments, the ASIC has a communication component that is adapted to transmit pressure data from the implantable assembly to the external device, wherein the external device has a communication component that is adapted to receive the transmitted pressure data, wherein the ASIC is adapted to transmit the pressure data upon receipt of an acknowledgment signal from the external device of receipt of a trigger signal from the implantable assembly.

Any of the features, systems, devices, and methods herein may be incorporated into other aspects of this disclosure unless specifically indicated to the contrary. For example, any of the calibration methods herein may be incorporated into any of the devices, systems, or assemblies described herein.

### BRIEF DESCRIPTION OF THE DRAWINGS

FIG. 1 schematically illustrates exemplary components of an exemplary implant.

FIG. 2 illustrate an exemplary implant with a flexible connector portion.

FIG. 3 illustrate an exemplary implant with a longer flexible connector portion than the exemplary implant in FIG. 2.

FIGS. 4A, 4B and 4C illustrates some exemplary views of an exemplary implant, which can be the same as or similar to the exemplary implant FIG. 2.

FIGS. 5A and 5B illustrate perspective sectional and front sectional views, respectively, of an exemplary first portion of an implant.

FIGS. 6A and 6B show side assembled and side exploded view of the exemplary first portion of an implanted device from FIGS. 5A and 5B.

FIGS. 7A, 7B and 7C illustrate an exemplary sensor portion of an implant.

FIGS. 8A, 8Bi, 8Bii, 8C, 8D and 8E illustrate an exemplary embodiment of an implant and an exemplary delivery device.

FIGS. 9A, 9B and 9C illustrate an exemplary implant, wherein the implant is adapted such that the sensor can rotate relative to the main housing about an axis, and the rotation axis is perpendicular relative to the main implant body.

FIGS. 9D and 9E illustrate merely exemplary antenna design and placement in any of the implants herein.

FIGS. 10A and 10B (side and top views, respectively) illustrate an exemplary implant that is adapted such that the sensor can rotate relative to the main housing about an axis, such that is can flex up or down relative to the elongate axis of the main housing.

FIGS. 11A and 11B (top and side views, respectively) illustrate an exemplary implant that includes a main body and a sensor.

FIGS. 12A-12G illustrate an exemplary implant that has a general square configuration.

FIG. 13 illustrates a portion of an exemplary implant in which a pressure sensor is hermetically sealed inside a fluid chamber.

FIGS. 14A and 14B illustrate that some exemplary implants can be coated with a biocompatible coating that may be optionally infused with weakly bonded to an anti-inflammatory agent or an anticoagulant.

FIG. 15 illustrates an exemplary implant that includes sensor and electronics mounted on an exemplary glaucoma draining device.

FIG. 16 illustrates an exemplary implant and an exemplary external device, and an exemplary communication protocol between the implant and external device.

FIG. 17 illustrates a merely exemplary schematic of operation of an exemplary autonomous intraocular pressure sensor system.

FIG. 18 illustrates exemplary implant locations, including but not limited to the anterior and posterior chamber, below the conjunctiva, and in Schlemm's canal.

FIGS. 19A and 19B (side and front views, respectively) illustrates the anatomy of a portion of the eye, illustrating exemplary locations for the one or more implants.

FIGS. 20A and 20B show human (a), and rabbit eye (b) to scale.

FIG. 21 illustrates a further exemplary schematic of operation of an exemplary autonomous intraocular pressure sensor system.

FIG. 22 illustrates an exemplary calibration plot of a piezoresistive pressure sensor in various environments.

FIG. 23 illustrates changes in pressure in response to voluntary blinking over time.

FIG. 24 illustrates RFID-Tag with interrogation and response signals.

FIG. 25 illustrates an exemplary in-vivo measurement of IOP in non-human primates

FIG. 26 illustrates an exemplary IOP sensing system, including an implantable housing that includes a calibration sensor.

FIG. 27 illustrates exemplary steps in a method that can factor in blinking-induced variations in IOP when determining a patient's IOP.

### DETAILED DESCRIPTION

This disclosure relates generally to intraocular pressure sensors, intraocular pressure sensing, and systems and assemblies for using, and the use of, the sensed pressure or information indicative of the sensed pressure. The sensors and methods herein may also, however, be used in sensing pressure in areas near or outside of the eye. For example, sensors and methods of use herein may be used in episcleral, cardiac or neural applications, including the brain.

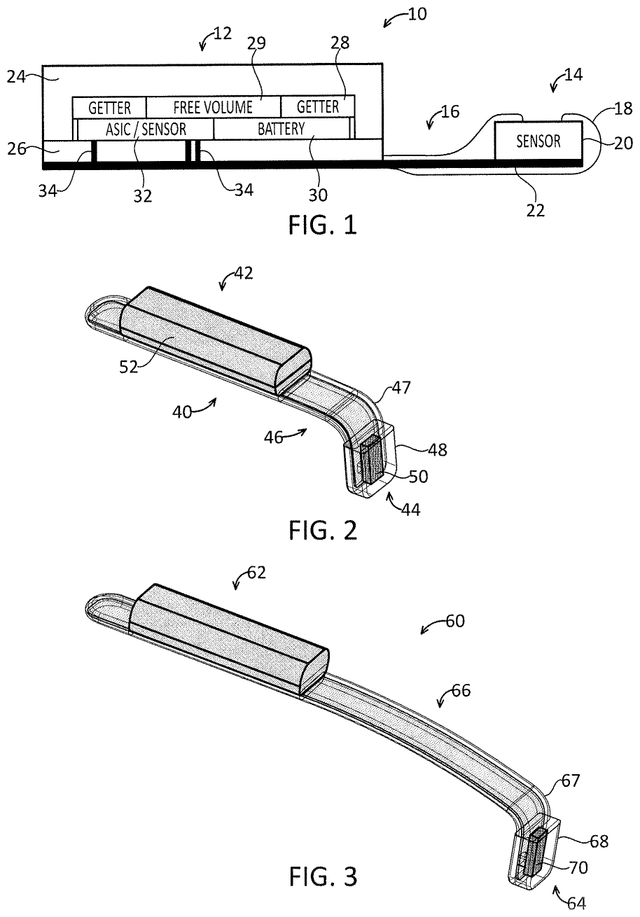

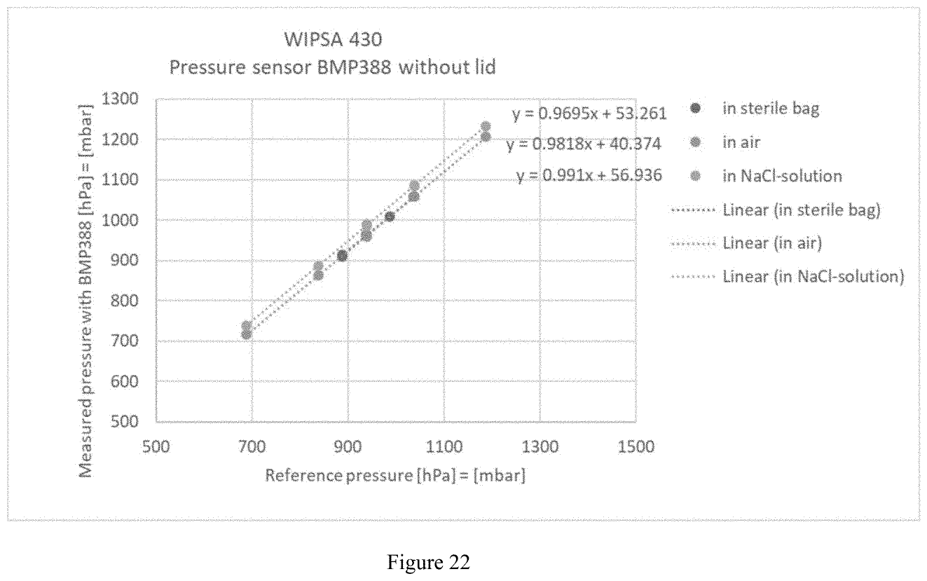

The first portion of the Detailed Description section herein and FIGS. 1-21 are included from PCT Pub. No. WO 2017/210316, which is fully incorporated by reference herein for all purposes. The first portion of the Detailed Description herein and FIGS. 1-21 may be incorporated into the disclosure that follows this portion and additional figures, to the extent that it is suitable to do so. For example, one or more devices, systems, assemblies, or methods in the first portion and/or in FIGS. 1-21 may be incorporated into one or more devices, systems, assemblies, or methods that follow the first portion and in FIGS. 22-26.

Some aspects of the disclosure include implantable intraocular pressure sensors that are adapted, configured, and sized to be positioned and stabilized within the eye and to communicate, optionally wirelessly, with one or more devices positioned within or outside the eye. A wireless intraocular pressure sensing device may be referred to herein as a “WIPS,” and an implantable device may be referred to herein an implant, or an implantable portion of a system or assembly.

Some of the devices, systems, and methods of use herein provide an exemplary advantage that they can sense intraocular pressure more frequently than possible with traditional tonometry and office visits, and can thus provide more frequent information regarding the change in pressure of an eye. For example, some devices herein are adapted to sense intraocular pressure continuously, substantially continuously, or periodically (regular intervals or non-regular intervals) when implanted in an eye.

An autonomous, implantable sensor is preferred in order to provide monitoring, optionally continuous, of IOP, in order to avoid relying on the patient to perform monitoring and management tasks that can be quite onerous for a sensor continuously recording IOP data. An autonomous implanted sensor can include an electrically operated sensor that measures pressure of the aqueous humor and converts it to an electrical signal, an internal power source, optionally provided by a rechargeable battery, an electrical controller such as a microcontroller or an ASIC to manage the electronic system, a memory unit comprising volatile and/or non-volatile memory, and a wireless link in order to, optionally, receive power wirelessly, download data to an external device, and optionally a data uplink to allow reprogramming capability. The data can be downloaded into a smart phone or a tablet that serves a data uplink to a caregiver's computer via a wireless or cabled network. Power can be provided from an external charging unit that has its own power management integrated circuit (PMIC), and may also have a wireless data transfer capability, and thus can function as an interface between the implanted device and the smart phone or a tablet.

FIGS. 1-17 and 21 illustrate aspects of merely exemplary implants that can be used with the systems and methods of use herein. FIG. 1 schematically illustrates exemplary components of an exemplary implant 10. Any of the implants herein can include a pressure sensor, a housing that hermetically surrounds an ASIC and battery, and a flexible substrate/connector to which the housing and pressure sensor are secured. The flexible substrate/connector can include an electrical connection to the pressure sensor and antenna. Any of the implants herein also include a calibration sensor, exemplary details of which are described herein.

One of the challenges when designing a wireless implant that includes an intraocular pressure sensor is conceiving of a way to incorporate components into a hermetically sealed device that includes a pressure sensor, antenna, power source, and controller, wherein the device can be implanted securely and safely into the eye, and still provide and communicate sensed data or information indicative of intraocular pressure to an external device.

Exemplary implant 10 includes first portion 12 secured to sensor portion 14 via connector portion 16. Substrate 22 extends between sensor portion 14 and first portion 12. Sensor portion 14 includes at least one pressure sensor 20 disposed within an encapsulation 18, optionally silicone or other similar material. Sensor 20 is in operable pressure communication with the external environment, such that external pressures can be transmitted to pressure sensor 20. This can be, for example, via an area of sensor portion 14 (e.g., encapsulation 18) that does not extend over the pressure sensor 18 as shown.

Substrate 22 carries electronics that allow signals from sensor 18 to be communicated to first portion 12. Data or signals indicative of sensed data can be communicated via sensor portion 14 to controller 32 with sealed vias 32 and 34, which is this exemplary embodiment comprises an ASIC. First portion 12 includes top casing 24 and bottom casing 26, which together form a hermetic seal that houses components therein. Top and bottom casings can be, in some embodiments, rigid glass material or titanium. The first portion also includes battery 30, and can also include water getter 28, and free volume 29.

FIGS. 2 and 3 illustrate substantially the same implants 40 and 60, with implant 60 having a longer flexible connector portion 66 than implant 42's connector portion 46. Both implants include a first portion 42/62, respectively, secured to the sensor portion via the flexible connector portion. Both implants also include sensor portion 44 and 64 respectively, which include sensors 50 and 70, respectively. First portions 42 and 62 can include any of the components of the implants herein, such as a power source, controller (e.g., ASIC), memory, water getter, etc.

Connector portions 46 and 66 each also include bend regions 47/67, respectively. Bend regions 47 and 67 are closer to sensor portions 44/64 than first portions 42/62. The bend regions are optional, as other embodiments do not necessarily need to include them.

In some embodiments the implant has an overall length such that the pressure sensor can be positioned in the anterior chamber and the housing is positioned in the suprachoroidal space of an average adult. The flexible substrate can include a bend, or region of increased curvature, as shown in some embodiments herein.

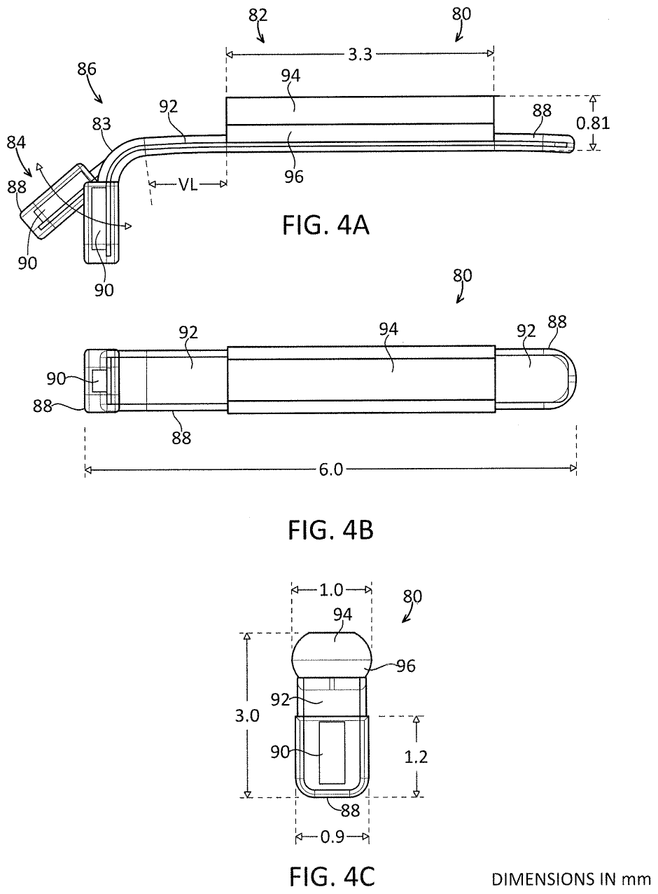

FIGS. 4A-4C illustrates some exemplary views of the exemplary implant, which can be the same or similar as implant 40 from FIG. 2, and which illustrate exemplary specific dimensions. The implants herein can be configured and sized to fit within a 0.6 mm to 2.0 mm outer diameter, and in particular a 1.0 mm outer diameter lumen, such as a needle. The dimensions shown in the FIGS. 4A-4C are illustrative and not limiting.

Implant 80 includes first portion 82, sensor portion 84, and connector portion 86. A casing or encapsulation 88 extends around sensor portion 84, connector portion 86, and along the bottom of first portion 82. Sensor portion 84 includes pressure sensor 90 disposed within encapsulation 88, but encapsulation can have a window therein so sensor 90 is in pressure communication with the environment. The first portion 82 can include any of the electronics and other components (battery, memory, antenna, etc.) described herein. Substrate or base layer 92 extends from the sensor portion 84 to the first portion 82, and carries electronics (e.g., flex circuits printed on a substrate) that electrically couple sensor 90 and electronics within first portion 82. Substrate 92 also comprises an antenna adapted for wireless data and power transfer.

As shown in the side view of FIG. 4A, the exemplary length of the housing of first portion 82 is 3.3 mm, whereas the height of the housing and encapsulation is 0.81 mm. As shown in the top view of FIG. 4B, the overall length of the implant is 6.0 mm. As shown in the front view of FIG. 4C, the overall width is 1.0 mm, while the exemplary sensor portion (including encapsulation) is 0.9 mm wide and 1.2 mm tall. The height of the overall device 3.0 mm.

FIG. 4A illustrate that connector portion 86 has a bend 83 along its length closer to the sensor portion 84 than first portion 82, and is flexible along its length, and the flexibility of connector portion 86 allows sensing portion 84 to move relative to first portion 82. In an at-rest, or nondeformed configuration, the bend 83 in connector portion 86 is such that connector portion 86 and sensor portion 84 have axes that are orthogonal to each other. Bend 83 can have a single radius of curvature of can have a varying radius of curvature.

Encapsulation 83 can be a deformable material such as silicone (compatible with off-the-shelf piezo and capacitive MEMS sensors). Top and bottom portions 94 and 96 can be glass or titanium, as is set forth herein.

The flexible electronics on the substrate can include the contacts for the sensor and the antenna. Incorporating an antenna into the flexible substrate is one way of incorporating an antenna into a compact implantable device while still allowing for data and power transmission.

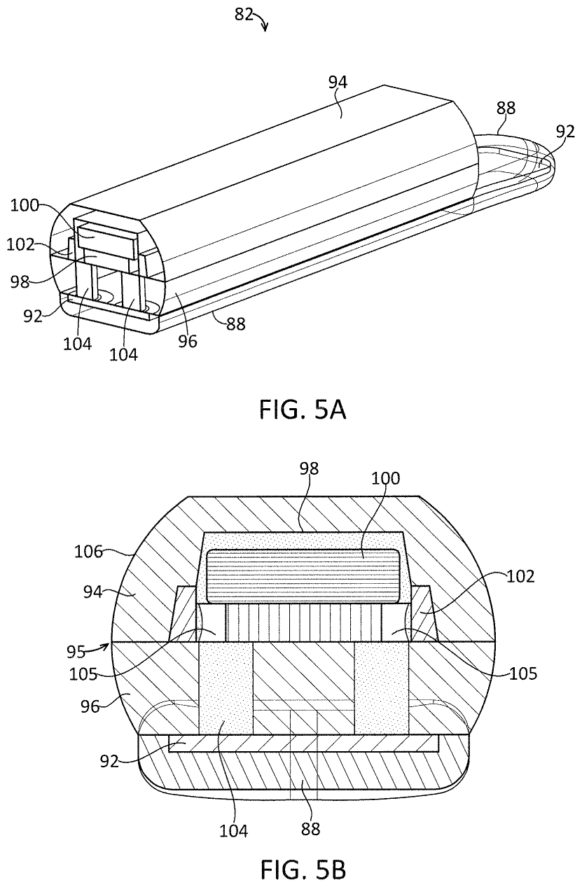

FIGS. 5A and 5B illustrate perspective sectional and front sectional views, respectively, of first portion 82. First portion 82 includes top and bottom housings 94 and 96, respectively, that interface at hermetic seal 95. The flexible electronics on substrate 92 are in electrical communication with vias 104, which are electrically coupled to housing electronics such as processor 98 (which can be an ASIC) and rechargeable battery 100. Optional water getter 102 is also disposed in the top portion of first portion 82.

First portion 82 also includes coating 106 thereon, which can be, for example without limitation, gold.

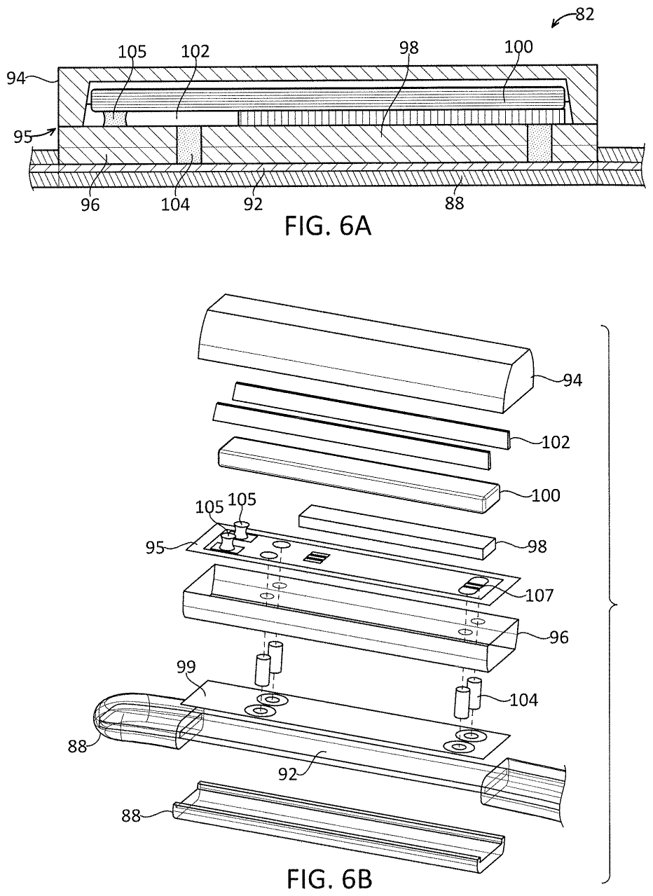

FIGS. 6A and 6B show side assembled and side exploded view of first portion 82 of an implanted device from FIGS. 5A and 5B. This first portion can be incorporated into any of the other embodiments herein. The relevant description of FIGS. 5A and 5B can similarly apply to FIGS. 6A and 6B. FIG. 6B illustrates more clearly the assembly and the manner in which the components are electrically coupled. The housing includes metallization 99, which provides an electrical connection with the flexible electronics on the substrate 92. Disposed between top housing 94 and bottom housing 96 is seal 95 and electrical connections 107, which are electrically coupled to vias 104. Connects 105 are in electrical communication with battery 100.

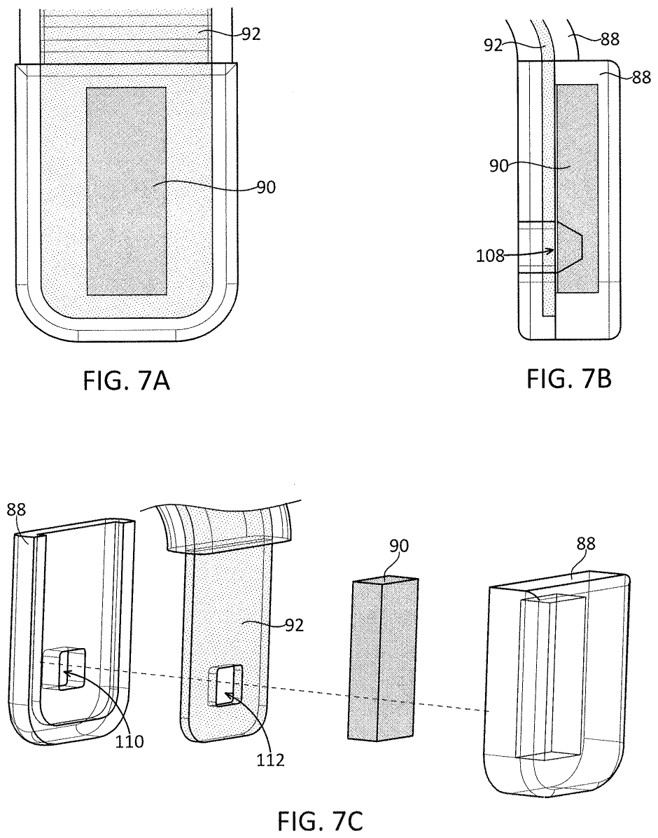

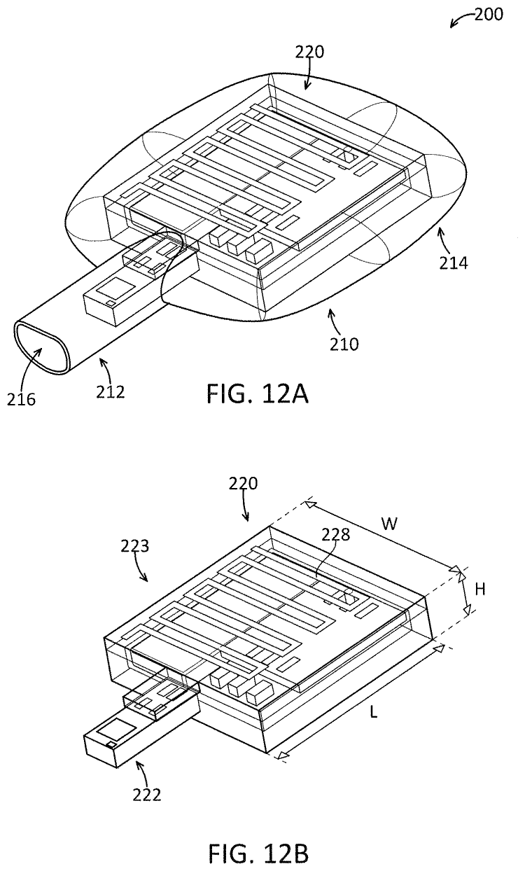

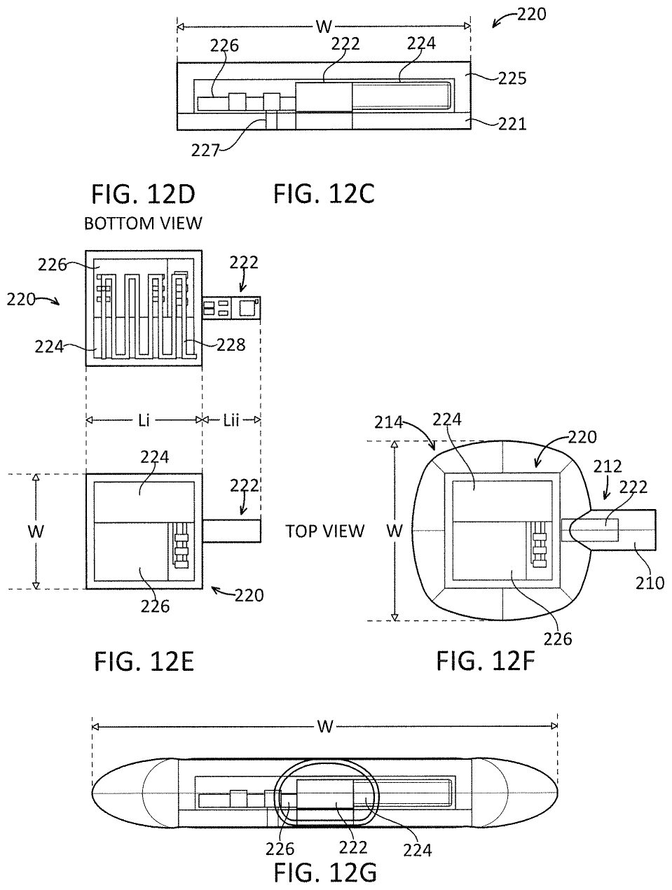

FIGS. 7A, 7B and 7C illustrate exemplary sensor portion 84 from FIGS. 4A-4C, but can be any of the sensor portions herein. FIG. 7A is a front view, FIG. 7B is a side view, and FIG. 7C is an exploded perspective front view. What can be seen is that encapsulation 88 and substrate 92 both include aligned windows or apertures therein, which allows the pressure sensor to communicate with the external environment. The windows together create opening 108 (see FIG. 12B) in the sensor portion. The windows may be filled with a material that allows pressure to be communicated to pressure sensor. The pressure sensor is “face down” on the flexible substrate and thus able to sense pressure via the access holes shown. The sensor electrical contact pads can be directly in contact with electronics on the flexible substrate, which can remove the need for wiring/wire bonding and requires an opening in the flex substrate and an opening in the encapsulation. Conductive lines/bond pads, and optional Parylene C coatings at piezo bridges are not shown in the figures, but can be included.

In any of the delivery procedures herein, an incision made in the eye during delivery can be 1 mm oval, or may be 1.2 mm.

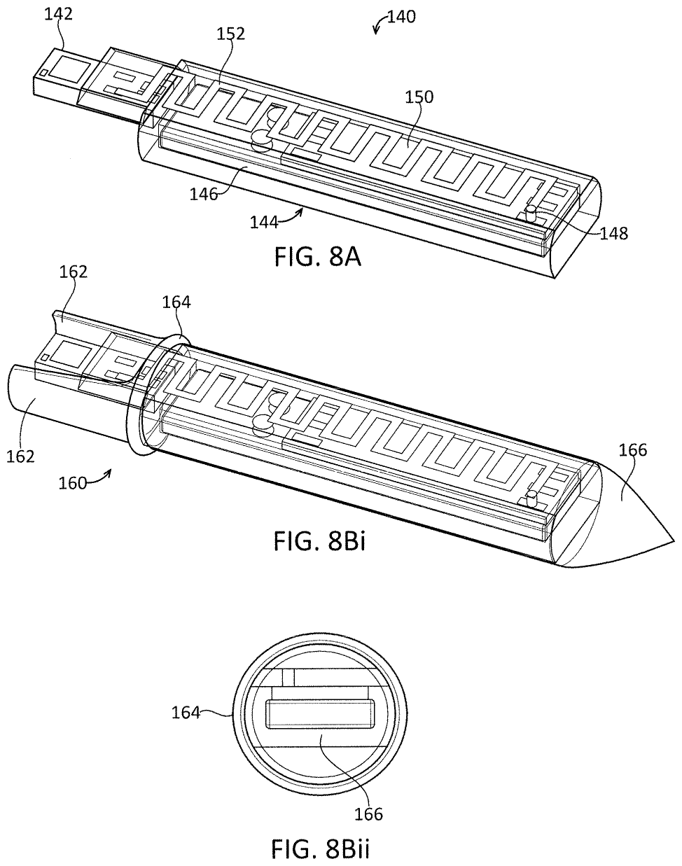

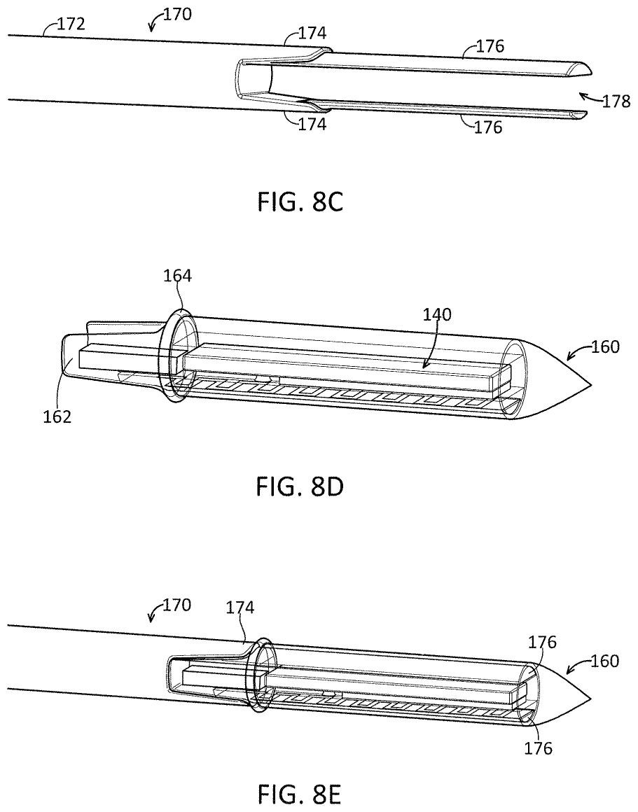

FIGS. 8A-8E illustrate an exemplary embodiment of implant 140 and exemplary delivery device. In this exemplary embodiment, the implant does not include a flexible elongate connector portion with a bend as in some of the embodiments above.

FIG. 8A shows a portion of implant 140. Sensor 142 is disposed at a first end of implant 140, and is coupled to housing 144. Housing 144 can include any components of any of the first portions herein. Housing 144 includes the encapsulation that encapsulates antenna 152, controller 150 (e.g., an ASIC), power source 146, and feedthrough 148 that connects ASIC 150 to the antenna 152. As in other embodiments herein, implant 140 can also include a metallic coating on the glass housing for hermeticity, one or more electrical lines on one or more glass or titanium substrates, an antenna ground plane, and a water getter (inside housing).

FIGS. 8Bi and 8Bii illustrate implant 140 from FIG. 4A but includes a biocompatible cover 160, optionally a polymeric material, including a plurality of sensor protective flaps 162 that extend at a first end (two are shown), a mechanical stop 164 for interfacing with a delivery device for insertion, and a conical second end 166 to ease the injection. Implant 140 is disposed inside cover 160, with two sides of sensor 140 protected by the flaps 162. Top and bottom sides of sensor 142 are not covered by cover 160.

FIGS. 8C and 8E illustrates an exemplary delivery tool 170 adapted and configured to interface with cover 160 (with implant 140 therein), which is shown in FIG. 8D, but inverted relative to FIG. 8Bi. Delivery tool 170 is adapted to facilitate the implantation of implant 140 and cover 160. Delivery tool 170 includes a main body 172 from which extend a first plurality of extensions 174 and a second plurality of extensions 176 (in this embodiment there are two of each). Extensions 174 are shorter than extensions 176 and are radially outward relative to extensions 176. One of the extensions 174 is aligned with one of the extensions 176, and the other of extensions 174 is aligned with the other of extensions 176. The plurality of extensions 174 interface with stops 164 of cover 160 when cover 160 is fully advanced within the inner space 178 of tool 170. Arms or extensions 162 on cover 160 are similarly sized and configured to fit within the space defined by arms 174. The radially inner arms 176 are positioned just slightly radially inward, and are sized and configured to be disposed within elongate channels within cover 160, which can be seen in FIG. 8E. In this embodiment body portion 172 of tool 170 has the same or substantially the same outer diameter as the cover 160. The elongate arms 176 can stabilize the relative positions of tool 170 and the implant during the delivery process.

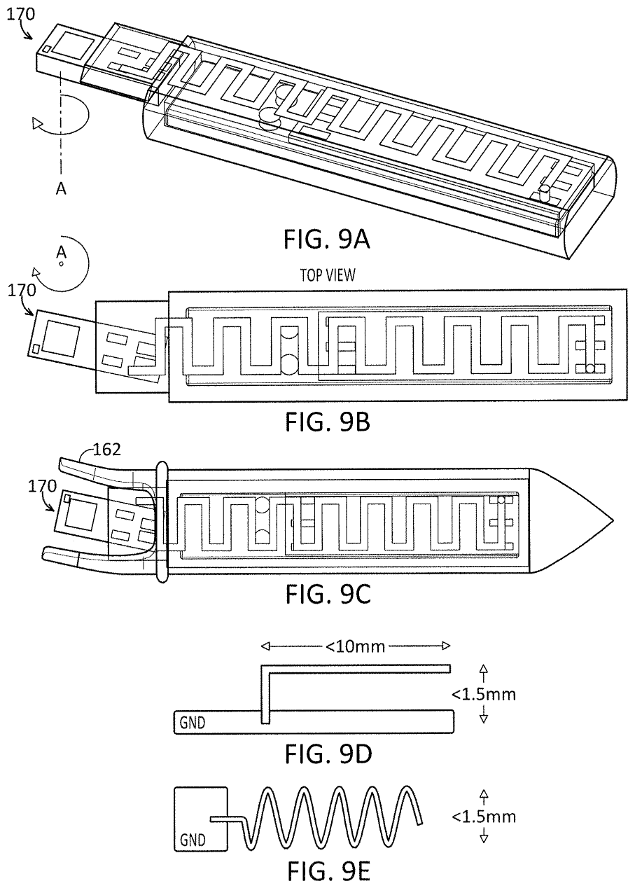

FIGS. 9A-9C illustrate an exemplary alternative embodiment to that shown in FIGS. 8A and 8B, but in this embodiment the implant is adapted such that sensor 170 can rotate relative to the main housing about axis “A,” and the rotation axis is perpendicular relative to the main implant body. All other components are described above and are not relabeled for clarity. FIG. 9A is a perspective view, and FIG. 9B is a top view. FIG. 9C is a top with cover, showing the two arms flexing with the rotation of the sensor. The protective cover follows the sensor orientation, as shown in FIG. 9C. In some embodiments the sensor can rotate up to 90 degrees, and in some embodiments no more than 45 degrees, such as 40 degrees or less, or 35 degrees or less, or 30 degrees or less, or 25 degrees or less, or 20 degrees or less, such as 12 degrees. In some embodiments the sensor is rotatable from 0 to about 90 degrees (e.g., 95 degrees). The implant in FIGS. 9A-C can be the same as the implant in FIGS. 8A-E in all other regards.

FIGS. 9D and 9E illustrate merely exemplary antenna design and placement in any of the implants herein. The antennas in the implant in FIG. 9A-9C can have other configurations and sizes as well.

Exemplary lengths for the implants shown in FIGS. 8A and 8A (without the cover) are 3-5 mm, such as 3.3 mm to 4.7 mm, such as 3.5 mm to 4.5 mm, such as 3.7 mm to 4.3 mm, such as 4 mm. Exemplary lengths for the covers herein, such as cover 160 from FIG. 8Bi are 4 mm to 6 mm, such as 4.3 mm to 5.7 mm, such as 4.5 mm to 5.5 mm, such as 4.7 mm to 5.3 mm, such as 5 mm. Exemplary widths for the implants shown in FIGS. 8A and 8A (without the cover) are 0.5 mm to 1.5 mm, such as 0.7 mm to 1.3 mm, such as 1 mm.

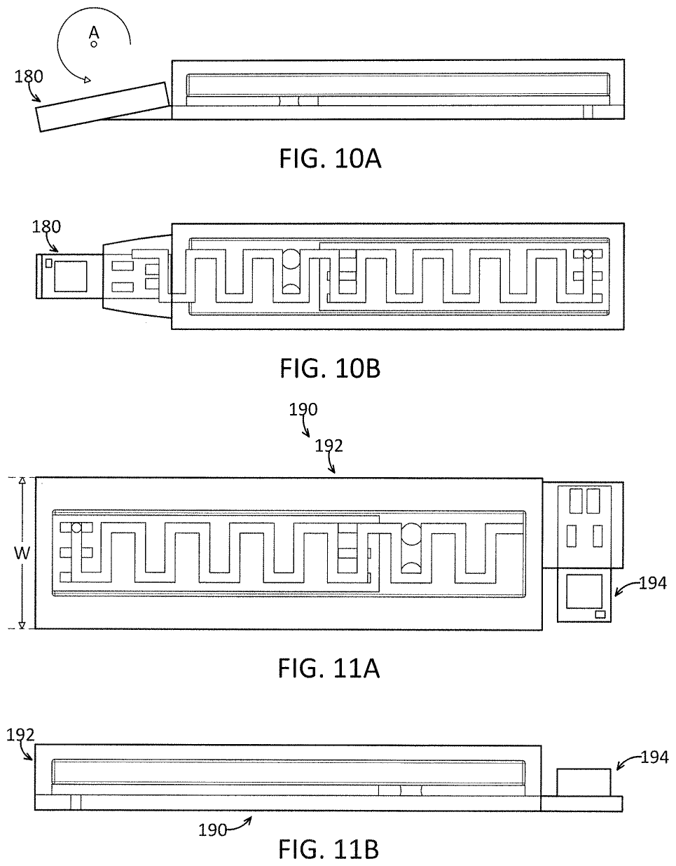

FIGS. 10A and 10B (side and top views, respectively) illustrate an alternative implant similar to that shown in FIGS. 9A-C, but in this embodiment the implant is adapted such that sensor 180 can rotate relative to the main housing about axis “A,” such that is can flex up or down relative to the elongate axis of the main housing. This embodiment may benefit from an angled sensor contact plane in the substrate.

FIGS. 11A and 11B (top and side views, respectively) illustrate an alternative implant 190, which includes main body 192 and sensor 194. Main body 192 can include any of the components set forth herein. Width W of the body 192 is wider than in FIGS. 9 and 10, and sensor 194 is oriented degrees relative to the sensor in the embodiment in FIG. 9A. Implant 190 can also be adapted such that sensor 194 can rotate with respect to main body 192. In some exemplary embodiments the sensor has a width that is about 0.3 mm to about 2 mm, such as from 0.5 mm to about 1.5 mm.

FIGS. 12A-12F illustrate an exemplary implant 200 that has more of a square configuration that embodiments above. At least a portion of the implant has more of a square configuration, even if there are one or more arms extending from a main body portion.

Implant 200 includes an outer cover 210 and internal portion 220. Any of the description herein relative to covers can also apply to cover 210, and any of the components described above can also be included in internal portion 220 (e.g., battery, processor, antenna, etc.). For example, internal portion 220 can include any or all of the components found in internal portion 140 shown in FIG. 8A, but they are organized within the implant in a different manner.

Figure is a bottom perspective view with the cover 210 on internal portion 220. FIG. 12B is the same view from FIG. 12A without cover 210. FIG. 12C is a front view of internal portion 220 without cover 210. FIG. 12D is a bottom view without cover 210. FIG. 12E is a top view without cover 210. FIG. 12F is a top view including cover 210. FIG. 12G is a front view including cover 210.

Internal portion 220 includes a main body portion 223 from which sensor 222 extends. The square configuration can make it easier to implant the implant in certain places in the eye. Main body portion 223 has a square configuration, with Length L and width W being the same dimensions. Body portion 223 can have, however, slightly rectangular configurations as well. Cover 210 similarly has a main body portion 214 with a generally square configuration and an arm portion 212 extending therefrom. Arm 212 has an open end defining lumen 216 so pressure sensor 222 can communicate with the environment.

Internal portion includes bottom housing 221 and top housing 225 (see FIG. 12C) that interface at a hermetic seal, examples of which are described herein. The internal portion also includes antenna 228 disposed in the bottom portion of the internal portion 220, battery 224, pressure sensor 222, processor 226 (e.g. ASIC), and electrical connect or via 227.

Other aspects of any of the embodiments herein can similarly apply to implant 200.

It is essential to provide a hermetic seal around the whole implant in order to ensure long term biocompatibility and also eliminate the risk of ocular fluids coming in contact with the miniature electronic circuit boards comprising the implant, potentially causing short circuits and other failures, including corrosion. In some embodiments, a hermetic seal may be formed by encasing the whole implant in a non-permeable material such as glass or Titanium, then closing the casing by means of laser welding, anodic bonding, or other types of sealing process that causes localized heating and fusion but does not cause a significant rise in temperature of the contents of the implant, for example, less than 2 degrees C. A challenge arises when designing a hermetic seal for a pressure sensor module, since it is necessary for the anterior humor of the eye to transmit its pressure to the sensor element inside the hermetically sealed implant in order to obtain reliable measurements of IOP.

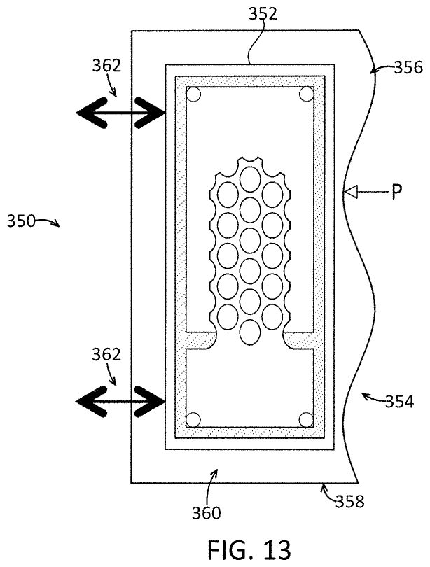

FIG. 13 illustrates a portion of an exemplary implant 350 in which pressure sensor 352 is hermetically sealed inside chamber 354. This concept of a fluid-filled chamber in which a pressure sensor is disposed can be incorporated into any implantable device herein. Chamber 354 includes a casing 358 and thin flexible membrane 356, which together define an outer wall of the implant. The implant also includes vias 362 that electrically connect pressure sensor 352 to other implant electronics, as described elsewhere herein. The chamber also includes inert fluid 360 contained within the chamber 354. Thin flexible membrane 356 is thin and flexible enough that it will transmit pressure P exerted by the anterior humor to fluid 360 within the chamber, which transmits the pressure to pressure sensor 352. In some embodiments flexible membrane 356 can be between 2 microns and 50 microns, such as 2-25 microns, such as such as 2-20 microns, such as 2-15, such as 2-10 microns, such as 5-10 microns. In some embodiments flexible membrane can be made of titanium or parylene. In some embodiments casing 358 can be made of titanium (e.g., TiN) or glass, and optionally coated with ceramic, examples of which are described herein. Examples of fluid 360 include, without limitation, nitrogen and silicone oil. The remainder of implant 350 can be the same as any of the other implants described herein.

In some embodiments the sensor comprises a piezoelectric sensing element where an inert fluid in the fluid chamber transmits hydrostatic pressure to the sensing element through the flexible membrane. In some embodiments the sensor comprises a capacitative sensing element wherein an inert fluid in the fluid chamber transmits hydrostatic pressure to the sensing element through the flexible membrane.

Any of the implants herein can have an unfolded length between about 2 mm to about 20 mm, such as between 2 mm and 15 mm, such as between 3 mm and 10 mm, such as about 7 mm. The housing can have a length of between 1 mm and 8 mm, such as between 1 mm and 7 mm, such as between 1 mm and 6 mm, such as between 2 mm and 5 mm, such as about 3 mm, or 3.3 mm.

The implants herein should be easy to surgically implant, and can optionally be implanted using a scleral tunnel or a clear corneal incision of perimeter less than 3.0 mm, optionally using a punch incision with a needle of outer perimeter preferably less than 1.2 mm, more preferably less than 1.0 mm. The implant should have long term biocompatibility, should not cause tissue erosion, should not cause the loss of corneal endothelium, and should not touch the iris, which will lead to deposition of iris pigment. The implants should provide a routine explanation option. The implants are preferably implanted in the sclera, or the conjunctiva, with the sensor being placed in the anterior chamber, posterior chamber, or inside the lens capsule as in the form of a capsular ring, while it may also be attached to an intraocular lens, the iris, the ciliary bodies, or be sutured to the ciliary sulcus.

In some embodiments the overall implant dimensions are less than 4.0 mm×1.5 mm×1.0 mm, preferably less than 3.5 mm×1.5 mm×1.0 mm, more preferably less than 2.5 mm×2.5 mm×1.0 mm, and most preferably less than 2.5 mm×2.5 mm×0.500 mm.

Any of the implants herein can have a folded length (after a portion of the implant is folded, or bent) between about 1 mm and 15 mm, such as between 1 mm and 12 mm, such as between 2 mm and 10 mm, such as between 3 mm and 9 mm, such as between 4 mm and 8 mm, such as between 5 mm and 7 mm, such as about 6 mm.

Exemplary pressure sensor dimensions can be 0.5 mm-1.5 mm×0.5 mm-2 mm. Off-the-shelf pressures sensors may be used in some embodiments.

Any of the implant housings herein, such as bottom housing 221 and top housing 225 in FIG. 12C (which may also be referred to as “casing” herein) can in some embodiments comprise glass or titanium with a gold or titanium plating (or any other biocompatible metal coating). The flexible connector, in embodiments that include one, can be a variety of suitable materials, such as, without limitation, a polymeric material encapsulated in a biocompatible silicone elastomer. The pressure sensor portion of any of the implants can include a sensor flexible membrane (e.g., Glass/Silicon), with other sides encapsulated in a silicone elastomer. In some embodiments the implant can have a parylene C coating on sensor membrane edges.

In any of the embodiments, any of the housings, such as a top housing or a bottom housing, can have a wall thickness of about 25-200 microns, such as about 50-150 microns, or about 75-125 microns, or about 100 microns. The wall thickness can provide hermeticity over a 10 year lifetime. Any of coatings herein can be about 0.1 micron to about 10 micron, such as about 0.1 micron to about 5 micron. The housings can comprise bonded top and bottom portions interfacing at a seal, as shown. The housings can have any of the following exemplary general shapes or configurations to provide a delivery profile that enables 1.0 mm external diameter: square, oval, circular, C-shaped, rectangular, chamfered, etc. The housings in FIGS. 5A and 5B, for example, have outer surfaces that are C-shaped, which allows the device to have a smaller profile than it would have with, for example, a more rectangular configuration.

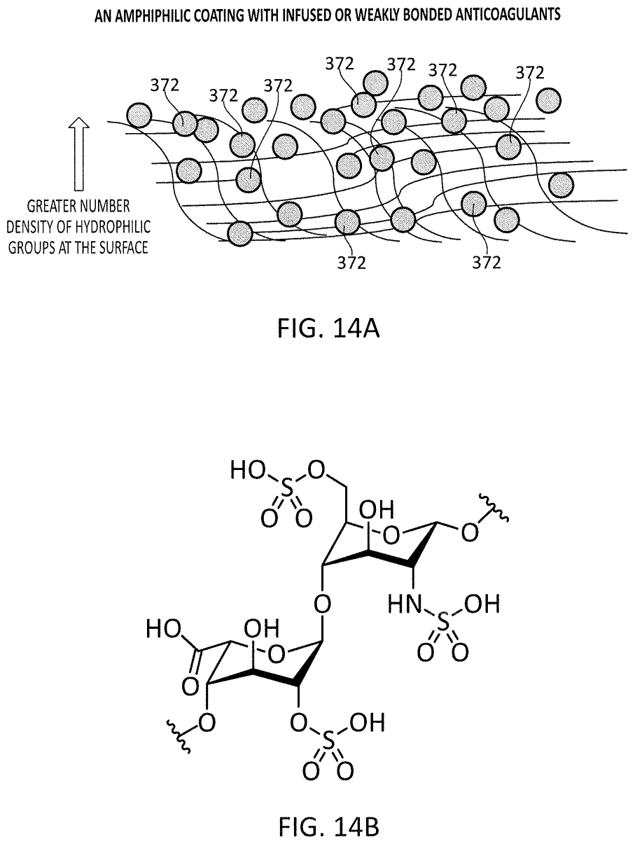

In some embodiments the implant is coated with a biocompatible coating that may be optionally infused with weakly bonded to an anti-inflammatory agent or an anticoagulant, which is illustrated in FIGS. 14A and 14B. The coating can be comprised of a cross-linked amphiphilic polymer with hydrophobic and hydrophilic segments. Typical polymers include hydrogels, silicone hydrogels and the like, with equilibrium water content ranging from 30% to 90% by weight. The cross-linked polymer comprising the coating folds such that the number density of hydrophilic groups increase towards the outer surface of the coating, while the surface contacting the implant may be richer in hydrophobic groups. This coating may include hydroxyl groups, amino groups, amides, sulfhydryl groups, thiols, as well as ionic moieties such as ammonium groups, alkyl ammonium groups and the like. These groups on the cross linked network comprising the coating are used to hydrogen bond or electrostatically bond anticoagulants such as Heparin sulfonate. FIG. 14A shows anti-inflammatory agents or anticoagulant groups 372, with the remainder of the groups being hydrophilic groups. An example of an anticoagulant is heparin, which is 13-20 kDa.

The hydrogel layer can have a gradient in number density of hydroxyl groups, wherein the gradient is in the opposite direction of the gradient in cross-link density.

The outer surface of the coating may be patterned or textured in order to promote fixation into the muscle in which the implant is positioned. The design of the texture is optimized to cause a minimal level of fibrosis causing adhesion of tissue to the implant without unduly enhancing immune response to the implant or chronic inflammation. Table 1 includes examples of components that may be included in such coatings.

TABLE 1

| Hydrophilic | Hydrophobic | Cross-Linking | |
|---|---|---|---|
| Monomers | Monomers | Agents | Anticoagulants |
| Hydroxyethyl | Methyl | Ethylene Glycol | Heparin |
| methacrylate | methacrylate | dimethacrylate | |
| Glyceryl | Styrene | Bis Acrylamide | Antithrombin |
| monomethacrylate | | | |
| Acrylic acid | Furfuryl | | Direct thrombin |
| | acrylate | | inhibitors |
| Methacrylic acid | | | lepirudin, desirudin, |
| | | | bivalirudin, |
| | | | argatroban. |
| Trimethylol propane | | | |
| triacrylate | | | |

Any of the power sources herein can be a battery or capacitor, such as a solid-state thin film battery, with an internal electrical connection to the controller, which can be an ASIC.

Any of the implants herein can have any of the following electronics: a controller such as an ASIC, electrical connections to sensor (such as flexible electronics on a substrate), hermetic via in a housing bottom portion, electrical connections to an antenna (such as flexible electronics on a substrate, and internal connections to the battery, and discrete electronic components (resistance, capacitance and/or inductance). In some embodiments that include an ASIC, the ASIC is ultra-low power to reduce the size of the overall implant.

In any of the embodiments herein, the ASIC can include a microelectronic circuit comprising a microcontroller, a flash memory, a non-volatile memory and a logic circuit. The logic circuit can include power management and data management modules. The ASIC can include a microelectronic circuit wherein said microelectronic circuit comprises conductive connectors of width in the range 36-360 nanometers.

Any of the implants herein can also include a H₂O getter, adapted to absorb moisture migrating through the housing to extend device lifetime with humidity below target 5000 ppm.

In some embodiments one or more components of the implant can be configured to correspond, or match, the curvature of one or more anatomical locations within the eye. This can lead to better compatibility within the eye.

The functionality of one or more components in the device can influence the overall size of the implant. For example, more battery power generally requires a larger battery size, which increases the size of the implant. Similarly, the size of an internal memory can increase as more memory is needed to store sensed data (e.g., temporarily). One or more ASICs can be used to manage the onboard components. It may be generally desirable to make the implant components as small as possible, but without sacrificing desired functionality. Determining how much sensed data is desired and/or the frequency of data sensing can thus influence the overall size of the implant.

In any of the embodiments herein, the antenna can have a space filling design, meaning that a maximum length of antenna is provided within a specific area, and wherein the antenna is connected to an electrical circuit that can be adjusted for its electrical impedance as a function of its resistive load. Examples of space filling antenna designs can be found in, for example, U.S. Pat. Nos. 7,148,850 and 7,026,997, the disclosures of which are incorporated by reference herein.

In any of the suitable embodiments herein, the antenna is disposed on a ceramic substrate disposed inside a housing, wherein the antenna has a thickness in the range of 100-500 microns.

In any of the embodiments herein, the circuit comprising the antenna can have a Q factor in the range of 10-50 under use conditions.

In any of the embodiments herein, the antenna includes vacuum deposited metal filaments on a ceramic substrate.

In any of the embodiments herein, the antenna has a conductive length of not less than 15 mm and not more than 100 mm.

In any of the embodiments herein, the antenna is adapted so that it transmits electromagnetic energy at a frequency that is not harmful to the human body.

Any of the implants herein can have more than one pressure sensor therein, or secured thereto.

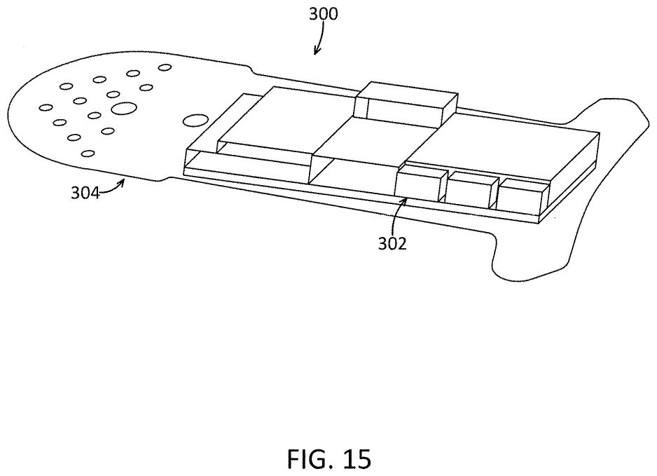

FIG. 15 illustrates an exemplary implant 300 that includes sensor and electronic 302 mounted on a glaucoma draining device 304, such as those manufactured by SOLX™. FIG. 15 illustrates a device that can both monitor pressure (using any of the electronic components and configurations herein in portion 303) and treat high IOP. Additional sensors can be implemented to detect oxygenation and proteins.

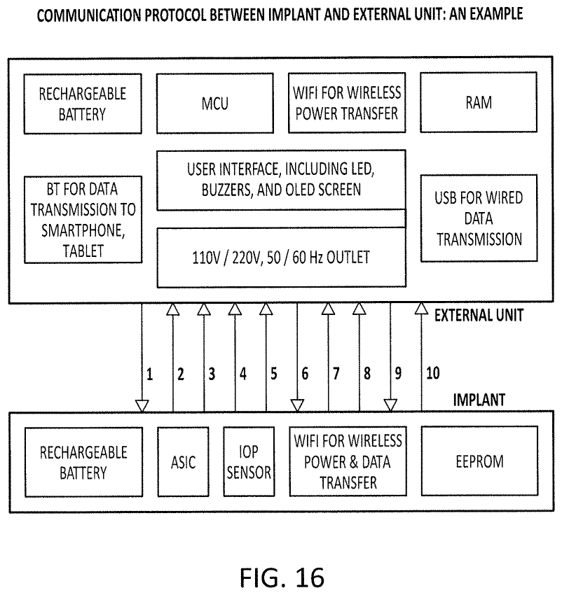

In any of the embodiments herein, the implant is adapted to sense IOP of an eye, or a portion of the eye. Any of the implants herein can include erasable memory. In some embodiments the system includes one or more external interrogation devices (“EID”s) that are disposed outside of the eye and can be adapted to communicate (preferably wirelessly) directly or indirectly with the implant. The EID is used to recharge the battery disposed in the implant, receive intraocular pressure data from the implant and reprogram the firmware embedded in the ASIC of the implant, when required. Communication between the implant and the EID follows a protocol, and example of which is shown in FIG. 16. This protocol involves encrypted data exchange, said encryption being compliant with all applicable Governmental regulations controlling confidentiality of medical information. Such a communication protocol also includes a handshake between the EID and the implant, the EID being the Master and implant being the Slave in this protocol. The exemplary protocol in FIG. 16 includes the following steps: 1) I am ready to transmit power and receive data; 2) I am ready to receive power, receive data, and I have data to transmit; 3) Transmission of data for initialization (code, time stamp, resonance frequency); 4) Data transmission (always recharging first step, when completed, data transmission (second step), when completed data transmission from External Unit to Implant (third step)); 5) Data transmission complete; recharging can begin in 2 seconds; 6) Wireless power transmission; 7) Threshold voltage reached, stop power transmission; 8) I am ready to receive data transmission (data for LUTs; reprogramming of firmware); 9) I have data/no data to transmit; 10) Data transmission, if step 9 gives code for data to transmit.

The one or more EIDs can receive information from the implant, such as pressure data (raw or processed) or other data indicative of pressure. The EIDs can also transmit information to the implant, such as instructions for programming or reprogramming some operational functionality of the implant (sensing software in the implant). One or more EIDs can also communicate with other EIDs, or external databases. An EID can also transfer power to the implant.

In some embodiments the system includes a patient EID (e.g., smartphone or a dedicated electronic device or an add-on device to a smartphone), which can be used or controlled by the patient. A patient EID can be used to charge the implant, receive data from the implant (e.g., by querying the implant), and optionally reprogram one or more algorithms stored in the implant. A patient EID can be wearable (e.g., wristband, watch, necklace) or non-wearable (e.g., smartphone, smartphone add-on, bedside device).

Systems herein can also include one or more physician EIDs, which can be wearable or non-wearable (e.g., dedicated electronic device, or laptop, smartphone or tablet add-on). For example, a physician can have access to one handheld EID (e.g., smartphone or tablet add-on), and have access to another medical personnel EID (e.g., a laptop computer with additional hardware and software capabilities). Any of the EIDs herein can be adapted to perform any of the EID functions described herein.

System software, on one or more of the EIDs, can be adapted to download and/or upload sensed pressure data, or information indicative or sensed pressure data to one or more EIDs or to the implant. System software includes software for data storage, data processing, and data transfer. System software can also facilitate communication between the patient EID and one or more physician EID (or other remote device).

The systems herein can also include one or more software and/or firmware applications to collect, compile, and/or store individual sensor data (e.g., sensor measurements) for diagnostic or treatment evaluation support by the medical personnel (e.g., ophthalmologist). The software and/or firmware may exist on one or more EIDs, or in some instances may be disposed on or more implantable devices. The systems herein can also include one or more software applications to collect and/or compile multiple sensors data as a basis for medical data analysis, allowing support for, e.g., predictive medicine.

Management of data can include processing of raw signals to, e.g., filter noise and enhance signal to noise ratio, application of algorithms that recognize and select a true pressure data from spurious signals, further processing of data to, e.g., recognize and document 1 hour to 30 day trends in pressure, and reprogramming of the ASIC and device firmware in response to specific data trends or command by caregiver.

Theoretically, a truly continuous monitoring of IOP requires continuous monitoring of IOP at a frequency exceeding the most rapid spike in IOP recorded (approx. 30 Hz). In reality, the data generated by such a sensor will be of such a magnitude that it will be difficult to manage even with frequent downloading of data, and will also require a large battery in order to manage the daily power consumption of such a device. In some embodiments an optimum amount of pressure data is therefore collected per day, based on patient needs, needs of treatment, upper limit of power available, and size of the memory units in the device.

In some embodiments the resolution and accuracy of IOP data range from 0.2 mmHg to 1.0 mmHg and form 0.5 mmHg to 2 mmHg, respectively. In some embodiments the frequency of data acquisition is minimum 2/day to maximum 1/15 min. In some embodiments the frequency of recharge is less frequently than 1/day. In some embodiments the frequency of data transmission to a caregiver can be once a day or more. In some embodiments wireless recharging and data exchange is performed using inductive coupling or electro-magnetic coupling among magnetic and/or electric antennas respectively, uses a body safe frequency and intensity, and with minimum attenuation by human tissue. The implants should have a 10 years life of battery, and have hermetically sealed package.

The sensed data and/or data indicative of the sensed data can be stored in one or more proprietary databases. In some embodiments all of the database information must be reviewed by a physician before being included in the database. In these embodiments the patients do not have access to the database. One or more databases can store time histories of sensed pressure measurements, or time histories of data indicative of sensed pressure.

The one more databases can include lookup tables with threshold pressures values, such that future sensed pressure data can be compared to the data in the lookup tables. The lookup tables can be for an individual or across a population of individuals. The lookup tables can be updated with new pressure data from one or more implants and one or more individuals. In some embodiments threshold levels can be a factor relative to therapy, optionally automatic drug delivery or a drug regimen. In some embodiments the sensed data can be used in a closed loop treatment loop. For example, pressure sensed over time can be input to a closed loop patient therapy protocol, such as closed loop drug therapy protocol.

The one or more remote databases can be a repository of all patient data, supplied by care givers, and encrypted; scalable; compatible with HIPPA regulations; and accessible to third parties

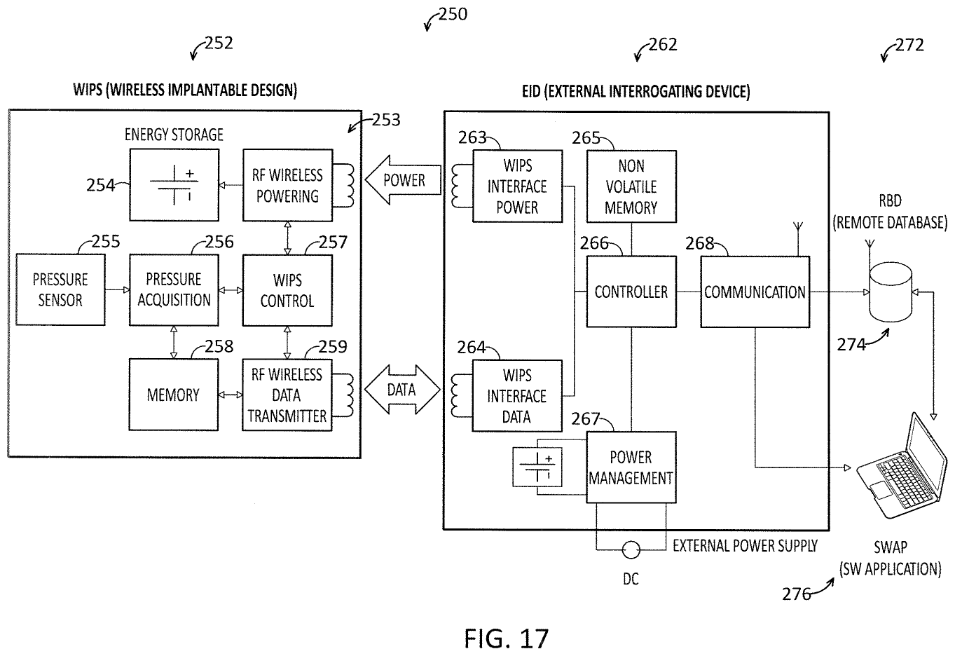

FIG. 17 illustrates a merely exemplary schematic of operation of an exemplary autonomous intraocular pressure sensor system. System 250 includes implant 252, one or more EID 262, remote database 274, and SWAP 276. Not all aspects of the system need to be included in the system. Implant 252 (which can be any implant herein), includes wireless powering device 253 (e.g., RF powering), energy storage 254 (e.g., rechargeable battery), processor 257 (e.g., ASIC), pressure sensor 255, pressure acquisition software 256, memory 258, and data transmitter 259 (e.g., RF data transmitter). EID 262 can provide power to implant 252, and can have directional data transfer with implant 252. EID 262 includes power interface 263, data interface 264, controller 266, non-volatile memory 265, power management 267, and communication module 268 (e.g., wireless comm module).

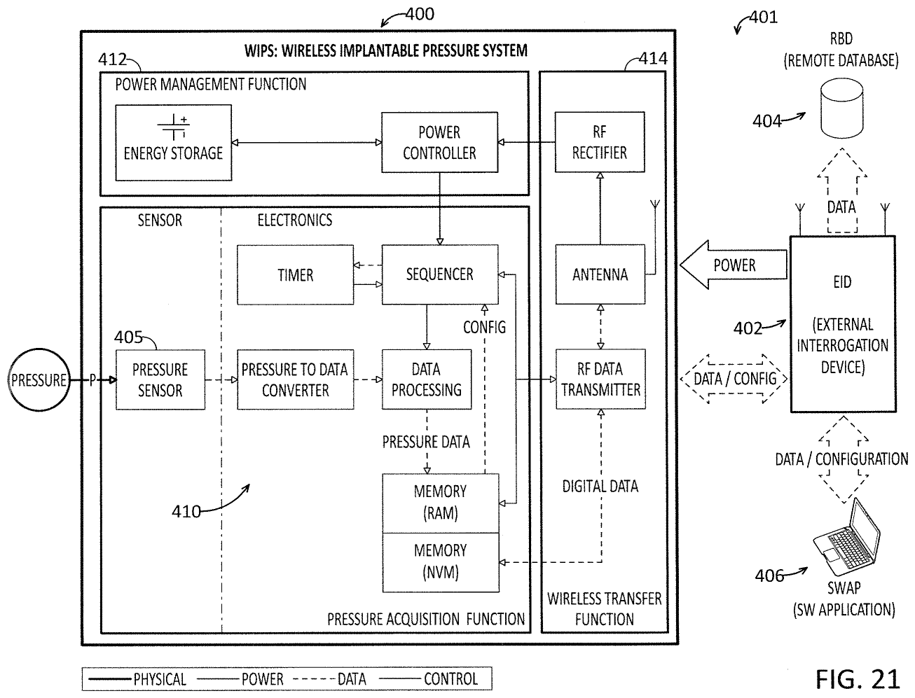

FIG. 21 illustrates a further exemplary schematic of operation of an autonomous intraocular pressure sensor system 401, including implant 400, EID 402, database 404 and SWAP 406. As shown, pressure sensor 405 senses pressure and sensed pressure or data is communicated to electronics 410. Power management 412 is in communication with wireless transfer function 414 and electronics 410. EID 402 can have any functionality described herein.

The disclosure herein also includes methods of delivering, or inserting, any of the implants herein. The disclosure herein also describes one or more surgical tools adapted for implanting the implant in or on the eye of a patient, and optionally a similar set of tools for implantation in animals for the purpose of validation studies. It is important that the implant, during delivery and after being implanted, not touch the corneal epithelium since the epithelial cells will be destroyed if they are touched.

The implantation of any of the implants herein in an eye will generally require one or more dedicated surgical tools and procedures. These implantation procedures will generally lead to minimal to no degradation of the patient's vision (e.g., by inducing astigmatism). In view of this, implantation through a needle (e.g., large gauge) is preferred over an incision. In some embodiments the entire implant is delivered through a needle. In some embodiments the needle is 13G needle, and in some embodiments it can be a 19-21G needle. An exemplary benefit of delivering through a needle is that no suturing is needed because no incision needs to be made.

Alternatively, the implantation of any implant herein can be combined with another surgical intervention, such as IOL implantation or in conjunction with other glaucoma drainage devices. In those embodiments, the implant and method of implant should be compatible with the incision already required for the implantation (e.g., IOL). In case of malfunction and/or risk to the patient, the implant is preferably also explantable with a similar, minimal invasive surgery, using dedicated tools. All tools and procedures are preferably compatible with both the right and left eye.

The implant is ideally positioned such as to not cause any visual obstruction, no degradation of any function of the eye, and generally not alter or aggravate the IOP of the patient (although some minor change in IOP may be caused). Additionally, in some embodiments, the implantation procedure does not deteriorate the vision of the patient by more than 0.25 diopters. An injection of the device (punch rather than incision) is preferred.

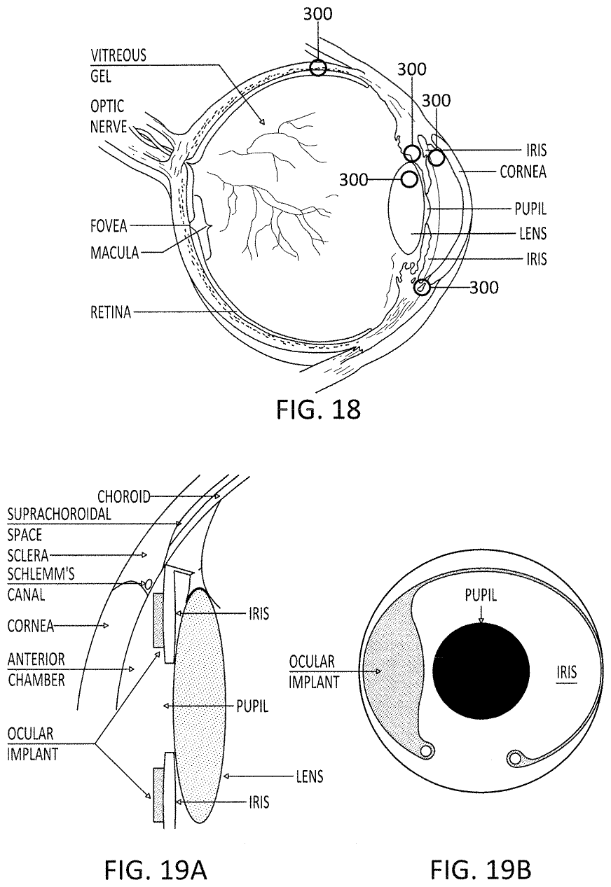

FIG. 18 illustrates exemplary implant locations 300, including but not limited to the anterior and posterior chamber, below the conjunctiva, and in Schlemm's canal. FIGS. 19A and 19B (side and front views, respectively) illustrates the anatomy of a portion of the eye, illustrating possible locations for the one or more implants. In some embodiments the implant includes two portions spaced from each other, and the implant is sized and configured such that the pressure sensor can be positioned in the anterior chamber while the implant housing is positioned in the suprachoroidal space. In some embodiments the implant is stabilized in placed due to, at least partially, the configuration of one or more components of the implant, and the interface with a portion of the eye. In some embodiments, fibrotic response can assist in keeping the implant, or a portion of the implant, in place.

Exemplary implantation procedures will now be disclosed. These exemplary procedures include an implantation of the sensor part of the implant in the anterior chamber angle, while the rest of the implant is positioned in the scleral/suprachoroidal space. These exemplary procedures include a punch incision and can be performed either at a slit lamp or in an operating room. The individual in which the implant is implanted is referred to generally herein as “patient,” but can include any person or animal, whether suffering from a medical condition or not. An eye may have more than one implantable device implanted therein. For example, it may be beneficial to have multiple devices in different locations to sense pressure at different locations within the eye, particularly if pressure varies from location to location within the eye.

A first exemplary procedure includes implantation through the conjunctiva. An eye is prepped with Betadine 5% sterile Ophthalmic solution. Topical anesthesia is then instilled to the surface of the eye. Lidocaine 1% preservative free solution is then injected under the conjunctiva in the area of insertion of the implant. The patient will then look opposite to the site of insertion (e.g., a patient looks up for insertion of the implant in inferior quadrants). The insertion device (e.g., needle) holding the sensor is entered through the conjunctiva approximately 3.5 mm from the limbus, into the sclera 2.5 mm from the limbus, and then directed to the anterior chamber angle. Once the sensor in observed in the anterior chamber, the needle is withdrawn and the tail of the implant will remain within the sclera with the sensor portion in the anterior chamber angle. The entrance of the needle will be watertight and there will be not be a need for suturing.

A second exemplary procedure includes implantation through cornea/paracentesis. An eye is prepped with Betadine 5% sterile Ophthalmic solution. Topical anesthesia is then instilled to the surface of the eye. Lidocaine 1% preservative free solution is injected in the anterior chamber. A paracentesis is then made opposite to the area of insertion of the implant. The insertion device then enters through the paracentesis and is advanced to the opposite angles, and the tail of the implant is inserted in the suprachoroidal space with the sensor portion of the implant remaining in the anterior chamber angle. The inserter is removed from the eye and the paracentesis is watertight and there is no need for suture placement.

When used in humans, the implantation of a wireless implant with sensor may be used to improve a patient's glaucoma treatment, either for early diagnostics or at the medication stage. The implants may also be used to gather data, whether in animals or humans.

Taking into account that patient compliance is one of the major challenge in IOP treatment, and in view of the average age of glaucoma patients, the periodic (e.g., regular) measurements of the IOP are preferably done with minimal patient actions (autonomously). The preferred implementation of this is through an active implant, which carries out measurements at optionally fixed time intervals utilizing an internal power source/power storage and internal memory/data storage, and is read out on a less regular basis by one or more EIDs, or alternatively with an EID which is capable of performing remote measurements at such a range that the patient is free in their movements and daily activities. In some embodiments the data transmission to physician EID can occur autonomously. For example, sensed data can be autonomously transmitted from the implant to a bedside EID at night, and then autonomously transmitted.

After implantation, the implant sensor senses pressure. Pressure can be sensed continuously (sensed during the entire time the implant is positioned in the patient, without interruption), or non-continuously. The implant can optionally have a continuous sensing “mode,” in which the implant is adapted to sense continuously, but the implant can also be taken out of the continuous mode, when switched to a different mode (e.g., no sensing, or a non-continuous sensing mode). When sensed non-continuously, it can be sensed periodically, either at regular intervals or non-regular intervals (e.g., sensed in response to detected events that do not happen with any known regularity). Exemplary regular intervals include one or more times a minute (e.g., 1, 2, 5, 10, 20, or 30 times a minute), one or more times a days (e.g., once, twice, five, twenty-four, 48 or 96 times a day). When sensed non-continuously, there may be epochs of time during which there is continuous sensing for a limited period of time, such as 1 minute of sensing, and then 59 minutes without sensing. An example of substantially continuous sensing is, for example, 30 times a minute. In some embodiments the pressure is sensed 1 time/day, or less (e.g., 1 time every two days). In some embodiments the frequency of sensing is between continuously and 2 times/day.

In some embodiments the implant is adapted to sense pressure at a particular frequency, but stores in memory only a subset of the sensed pressures. Sensed data can be stored in, for example, a first in first out manner.

The required IOP measurement pressure range can be, in some embodiments, 1 mmHg around ambient pressure and up to an overpressure of approximately 50 mmHg above ambient pressure.

The recorded data can be stored in a memory and transmitted periodically to an ophthalmologist (e.g., EID) for treatment evaluation. It may be beneficial for the patient not to have direct access to the IOP data. In some embodiments, in which the patient has an EID, the patient's EID is adapted to do one or more of the following: retrieve stored IOP data from the IOP implant; retrieve operational status of the implant and any error messages; and transfer power to the IOP implant to charge the power storage component.

In embodiments in which an IED provides power and data transfer to the implant, they are both preferably achieved wirelessly, typically over an RF link. The EID can receive this data and status of the implant, and communicate it to the ophthalmologist (or other second EID) for treatment evaluation support. In addition, the data collected by any or all EIDs can be compiled in databases, optionally in an anonymized format, in order to use the collective patient data to support applications in predictive medicine and e-health.

In embodiments in which medical personnel have access to an EID, that EID can be adapted to perform the same tasks as the patient EID, but it may additionally be adapted to perform any of the following: program some basic operational functions of the implant (e.g., measurement interval), and allow calibration of the implant's IOP values against e.g., a traditional tonometer.

In some embodiments an external interrogation unit has a resonant circuit for wireless charging of the implant; ASIC for power and data management; can be mounted in furniture, bed, eyeglasses for close access to the implant coil; adapted to reprogram the firmware, algorithm in the implant; can have multiple units for patient convenience; and can be portable.

Sensor readings from one or more implants may need to be calibrated based on, for example, their position in the eye. In some embodiments the position of the one or more wireless IOP sensors is such that the pressure reading at the sensor is directly linked to, or can be calibrated back to, the fluid pressure in the anterior chamber. Currently, intraocular pressure is measured by a device applying a force to the anterior surface of the cornea. It may be that sensor readings sensed within the eye, or even at different locations within the eye, result in pressure sensor readings that are different than are currently measured at the anterior surface of the cornea. Sensor readings obtained with implants herein may thus need to be calibrated with existing pressure readings taken at the anterior surface of the cornea. Different sensor locations may also need to be calibrated individually, particularly if sensor readings are different at different locations within the eye. Additionally, pressure readings may be more accurate or provide more reliable information at particular locations within the eye.

Patient to patient variability, which can be variability across the board or at particular locations, can require calibration and/or recalibration for each patient.

In some embodiments more than one sensor may be implanted in an eye, and the different sensors may obtain unique sensor readings. The system can be adapted to use the different sensor data to, for example, provide a pressure difference between two sensors, and improved patient therapy or diagnostics.

In some embodiments, in order to use the collected pressure data (patient-specific or anonymized), a remote database (e.g., cloud database) of the recorded IOP values exists. The database can interact with one or more EIDs and/or clinicians, and can be used to process the IOP data.

While the implant generally only communicates when interrogated by an EID (due to power constraints), in some modified embodiments the implant may be adapted with sensed data event detection, generally requiring a processing component. For example, when sensing pressure, the implant can be adapted to detect a threshold pressure or other event. The event detection can trigger a variety of actions, such as, for example, automatic drug delivery, storing future sensed data after the detected event, and automatic transmission of data to one or more EIDs.

In some embodiments the implant and one or more EIDs can be adapted so that the one or more EIDs can reprogram one or more functions of the implant. For example, an implant's sensing frequency, event detection, sensed threshold value, etc., can be reprogrammed by the one or more EIDs. Reprogramming can occur in response to a change in the database lookup tables, for example. Reprogramming can also occur in response to data sensed from the particular patient.

Any of the implants herein can have an internal power source that can be recharged using an EID. In some embodiments charging is done via an inductive or electromagnetic coupling with emitted powers from the EID in the 10-30 mW range, such as 25 mW, or in the range of 1 W to 5 W, such as 3 W. In some embodiments the EID can transmit power and data to the implant.

In some embodiments the length of the antenna in the implant is 30 mm or less, such as 25 mm or less, such as 15 mm or less, such as 10 mm or less, and a height of 3 mm or less, such as 2.0 mm or less, such as 1.5 mm or less.

This exemplary power transfer data shows feasibility for these antenna designs, with the exemplary coiled antennas more efficient than the straight antenna. Initial prototypes have used the MIL-STD 883 for hermeticity requirements. The norm specifies 5000 ppm of H₂O vapour as upper limit. Rationale: 5000 ppm is condensation point of water vapour at 0 deg C. With less than 5000 ppm of H₂O, water will never condensate: above 0 deg C. it is vapour, below 0 deg C. the condensed water will freeze. No liquid water can be present below 5000 ppm at any temperature. Note: At eye temperature, the dew point is much higher than 5000 ppm, namely 25000 ppm.

The following describes some optional features of any of the implant housings (e.g., around a battery and ASIC) herein: Any of the implants herein can achieve <5000 ppm H₂O over a 10 year lifetime. There may be a trade-off between housing thickness and permeability: thicker housing walls provide lower permeability but cause a larger implant volume. A larger inner volume gives more allowed H₂O before reaching 5000 ppm but for larger implant volume. It may be preferable for the housing material for electronics and battery to be glass, ceramic or metal (Ti) or any metal/glass/ceramic combination. Additional conformal barriers like Parylene C are also considered. Any of the implants herein can include a H₂O getter. H₂O getter can be a solid/polymer that binds H₂O molecules entering implant, lowering internal H₂O pressure (until full). The H₂O getter can extend lifetime below 5000 ppm at a given permeability.

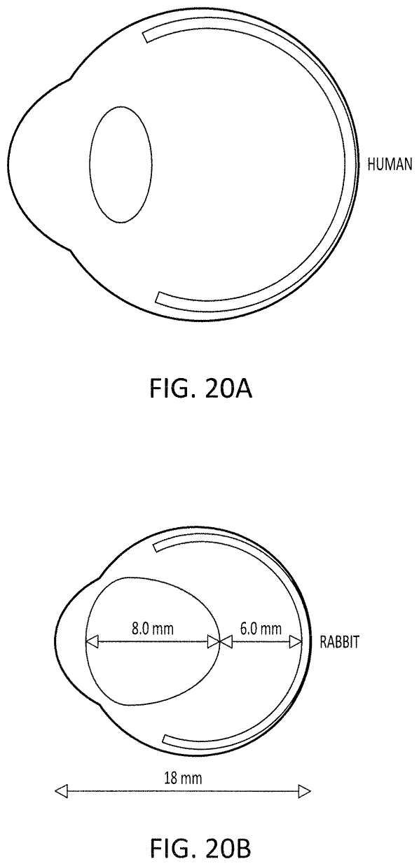

The disclosure herein includes methods of use in animals (e.g., rabbits, mice, rat, dog) aimed at initial IOP data collection and serving for validation studies for humans or veterinary applications. The disclosure herein also includes human uses, which can be aimed at collecting regular patient IOP values to be used for any of diagnostics support, drug selection support, and evaluation of patient compliance to glaucoma treatment. The rabbit eye is a standard biomedical model for validating human intraocular implants as it has similar dimensions (see FIGS. 20A-20B), but shows accelerated fibrotic and inflammatory behavior with respect to human eyes. Any of the WIPS herein can thus be implanted in rabbit (or other animal) eyes. The implantation of implantable device in animals can provide any of the following: data can be gathered for glaucoma pharmaceutical development programs; data collected by a device in a rabbit's eye can be used as clinical evidence for a future human product; and valuable usability inputs can be generated.

FIGS. 20A and 20B show human (a), and rabbit eye (c) to scale, including schematic representation of the lens (yellow), retina (red) and vitreous and aqueous bodies (blue).

An IOP device that is implanted in a rabbit should therefore, in some uses, be the same or nearly the same as a current or future human device. Some difference between rabbit implants and human implants may include one or more of: the implant location in a rabbit eye may be different than in the human eye in view of the dimensional differences of anterior and posterior chamber of a human vs. rabbit eye (the location should be, however, medically representative (IOP, fibrosis, inflammation)); the implantation time may be shorter with the rabbit compared to the human application; the surgical tools may differ in size to match the dimensions of the rabbit's eye, but not in function compared to the tools for human implantation; and the regulatory requirements that apply for rabbit implantation may differ from those for human implantation. All other aspects can be the same as those of human implants described in the following section.

The system and implants herein can also be used for research purposes to investigate changes in intraocular pressure due to certain activities, such as exercise, or sleep, or drug therapy.

Additional Examples. The following are additional examples of the disclosure herein.

An optionally autonomous, wirelessly connected, intraocular pressure sensing implant, wherein said implant is less than 3.5 mm in its longest dimension.

The implant of any of the additional examples herein wherein said implant has an internal rechargeable power source that can provide operating power for at least one half day (12 h) of operation.

The implant of any of the additional examples herein wherein said power source is a rechargeable battery.

The implant of any of the additional examples herein wherein said implant has power and data management integrated circuits that consume less than 50% of its stored power in resistive losses.

The implant of any of the additional examples herein wherein said implant utilizes at least one application specific integrated circuit for power and data management.

The implant of any of the additional examples herein wherein said implant comprises a sensor that senses intraocular pressure and collects pressure data more than once every 12 hours and no more than once every minute.

The sensor of any of the additional examples herein wherein said sensor operates at a frequency of 30 Hz or more.

The implant of any of the additional examples herein wherein said ASIC is controlled by firmware that is reprogrammable by an external unit via wireless communication of data subsequent to implantation of any of the implants herein.

The implant of any of the additional examples herein wherein said ASIC downloads data to said external unit that is programmed to receive said data.

The implant of any of the additional examples herein wherein said ASIC actuates commencement of wireless recharging from said external unit upon receipt of a trigger signal.

The implant of any of the additional examples herein wherein a trigger signal may be transmitted from an external unit.

The implant of any of the additional examples herein wherein said trigger signal may be generated inside said ASIC when the output voltage of said rechargeable battery of claim 3 drops below a threshold voltage that is above the voltage at which the battery shuts down.

The implant of any of the additional examples herein wherein said implant is rendered biocompatible by being hermetically sealed.

The implant of any of the additional examples herein wherein said sensor is periodically actuated by an ASIC.

The implant of any of the additional examples herein wherein a trigger can be externally or internally generated.

The implant of any of the additional examples herein wherein a trigger signal when internally generated, is reprogrammable.

The implant of any of the additional examples herein wherein data is processed and filtered in firmware in an ASIC.

The implant of any of the additional examples herein wherein data is further processed, analyzed and encrypted in a data processing module in an external unit.

The implant of any of the additional examples herein wherein data is downloaded to a smart phone or a tablet or a dedicated electronic device (e.g., the EID).

The implant of any of the additional examples herein wherein data is transmitted from an EID, a smart phone or a tablet to the computer of the caregiver.

The implant of any of the additional examples herein wherein data is transmitted by the caregiver to a remote data base.

An implant sized to be stabilized within an eye, the implant comprising an intraocular pressure sensor.

An implantable intraocular pressure sensor, comprising a pressure sensor and electronics coupled to the pressure sensor.

Any of the claimed implants, adapted to be positioned in any of the anatomical shows or described herein.

A method of positioning an intraocular pressure implant, comprising a sensor, in an eye.

A method of sensing intraocular pressure continuously, substantially continuously, or periodically, with an implantable intraocular sensor sized and configured to be stabilized within an eye.

Any of the claimed methods, further comprising transmitting information, either pressure data (e.g., raw or processed) or information indicative of pressure data wirelessly to an external device.

Any of the methods of calibrating an implantable pressure sensor herein.

A method of sensing pressure in an eye with an implantable device, wherein the implantable device is adapted to process the sensed pressure.

The implant of any of the additional examples herein wherein the implant comprises a memory module that further comprises non-erasable and/or reprogrammable memory elements.

The implant of any of the additional examples herein wherein the implant comprises a controller that controls its pressure sensing, data collection, processing, storage and transmission, and recharging operations.

The implant of any of the additional examples herein wherein a wireless connection between said implant and an external unit is operated at below 6 GHz, e.g., at 868 MHz, 900 MHz or 2.4 GHz.

The implant of any of the additional examples herein wherein the wireless connection between implant and external unit comprises electro-magnetic or inductive coupling between a transmitting and a receiving antenna.

The implant of any of the additional examples herein wherein the wireless connection between implant and external unit utilizes one or more antennas which can be e.g., straight, coiled, or flat.

The implant of any of the additional examples herein wherein the wireless connection between implant and external unit coupling has a system Q factor not less than 10 and not exceeding 100.

The implant of any of the additional examples herein wherein a transmitter coil transmits wireless power not exceeding 25 milliwatts.

The implant of any of the additional examples herein wherein recharging of the implant occurs at any distance between 2 cm and 2 meters.

The implant of any of the additional examples herein wherein preferred modes of charging the implant are either at 2-5 cm over 1 hour or 0.5-2.0 meters over 8 hours.

The implant of any of the additional examples herein wherein data is transmitted by the EID, the patient's smartphone or tablet to a remote data base.

Any of the devices, systems, and methods described below may integrate and incorporate any of the disclosure above unless specifically indicated to the contrary. For example, any of the devices below that incorporate a second sensor (including the use of a second sensor) or any of the calibration concepts below may incorporate any of the aspects of the disclosure above (e.g., devices, systems, features, methods of use) unless specifically indicated to the contrary.

Some of the devices, systems, and methods of use herein provide an exemplary advantage that they can sense intraocular pressure more frequently than possible with traditional tonometry and office visits, and can thus provide more frequent information regarding the change in pressure of an eye. For example, some devices herein are adapted to sense intraocular pressure continuously, substantially continuously, or periodically (regular intervals or non-regular intervals) when implanted in an eye.

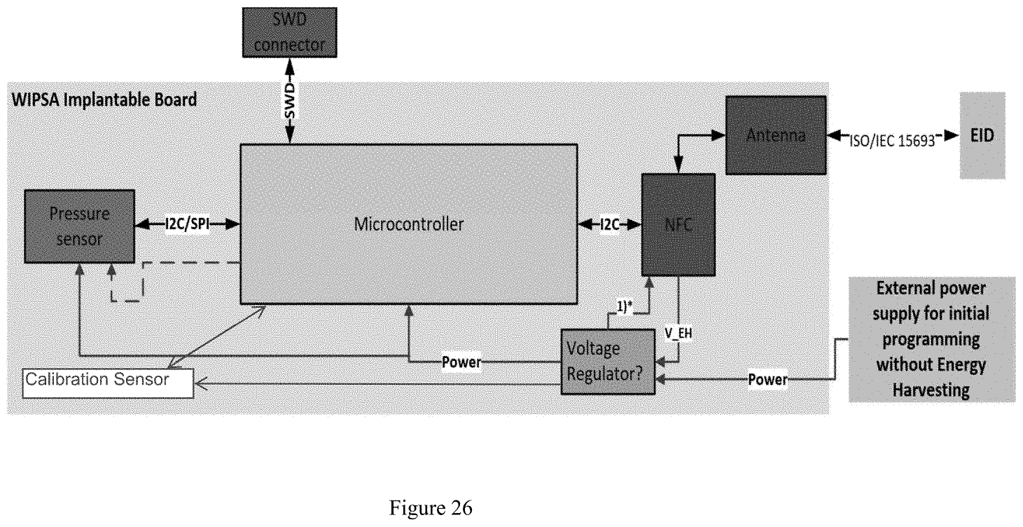

The pressure sensing systems herein can be autonomous, implantable sensors that are adapted to provide monitoring, optionally continuous, of IOP (or sensed data/electrical output signals indicative of IOP), in order to avoid relying on the patient to perform monitoring and management tasks that can be quite onerous for a sensor continuously recording IOP data. An autonomous implanted sensor can include an electrically operated sensor that measures pressure of the aqueous humor and converts it to an electrical signal, an internal power source, optionally provided by a rechargeable battery, an electrical controller such as a microcontroller or an ASIC to manage the electronic system, a memory unit comprising volatile and/or non-volatile memory, and a wireless link in order to, optionally, receive power wirelessly, download data to an external device, and optionally a data uplink to allow reprogramming capability, an exemple of which is shown in FIG. 26. The data can be downloaded into a smart phone or a tablet that serves a data uplink to a caregiver's computer via a wireless or cabled network. Power can be provided from an external charging unit that has its own power management integrated circuit (PMIC), and may also have a wireless data transfer capability, and thus can function as an interface between the implanted device and the smart phone or a tablet.

Pressure sensors generally record absolute pressure, in other words, the actual pressure being applied by the aqueous humor on the sensing surface of the sensor. IOP, defined as the difference of pressure exerted by the aqueous on ocular tissue and the ambient pressure of the atmosphere. Therefore, it is necessary to record the ambient pressure when the implanted sensor records the pressure of the aqueous humor, preferably at the same time and at the same place. In some embodiments, an atmospheric pressure sensor may be included in the electronic design of the external interrogation device and programmed to record ambient pressure at the same time as the implanted sensor records pressure of the aqueous humor.

In some embodiments, an additional (e.g., second) sensor may be incorporated into or on the implant housing, wherein the second sensor is adapted to sense an amount of fibrous tissue being deposited on the sensing surface of the sensor post implantation, or at least provide an amount that is indicative of an amount of fibrous tissue that has deposited onto the implant housing. Any of the second sensors herein may be referred to herein as a calibration sensor. For example, the additional sensor can be positioned on a printed circuit board (“PCB”) of the implantable housing. In some exemplary embodiments, this additional sensor may be a mass sensor such as, for example without limitation, a quartz microbalance (“QCM”) or a surface acoustic wave (“SAW”) sensor that is adapted to sense the amount of fibrous tissue being deposited on the sensing surface of the sensor subsequent to implantation. While post-operative inflammation must be kept at a minimum through the use of biocompatible materials as coatings applied on the implant surface, it is impossible to eliminate post-operative inflammation completely, especially inflammation caused by wound healing subsequent to surgery. Common two-port SAW devices can typically include a piezoelectric substrate (e.g., ST-cut quartz) having two metallic interdigital transducers (“IDT”) deposited on its surface. Applying an electrical signal to one of the IDT triggers a mechanical acoustic wave on the surface that is re-transformed into an AC signal on the second transducer. In contrast to this, RFID-Tags typically include a SAW sensor with only one IDT and a distinct reflector pattern leading to time-dependent signal modulation that is suitable for identifying individual devices. Generally, the resonance frequency of surface acoustic wave devices is determined by the structure width of the IDT.

Δf=k₁f₀²tρ=k₁f₀Δm/A (Equation 1).

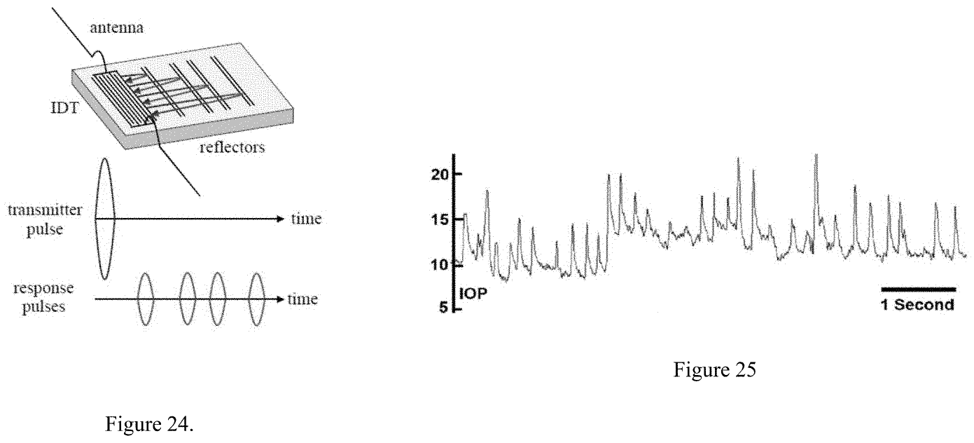

In this equation, Δf is the shift in the resonance frequency of the SAW sensor, f₀ is the resonance frequency, typically between 10-1000 MHZ, k₁ is a material constant, A is the area of the sensor surface, and m is the mass of the fibrous deposit, as shown in FIG. 24. A RFID tag may be provided with its own antenna, or it may be connected to a single antenna assembly that can be used for data and power transfer between the implant and an external interrogation device (“ED”).

One aspect of this disclosure is an implantable intraocular pressure sensing device, such as any of the implantable devices herein. The device can include an implantable housing that can include an intraocular pressure sensor and a calibration sensor, the calibration sensor adapted to create an output signal that is used by any of the methods herein to calibrate an output signal from the intraocular pressure sensor. FIG. 26 illustrates a merely exemplary schematic representation of an exemplary pressure sensing system, wherein an implantable housing includes a microcontroller, pressure sensor, and calibration sensor, among other components. The calibration sensor may be a mass sensor, such as a quartz microbalance. The calibration sensor can be a surface acoustic wave (“SAW”) sensor, such as a two-port SAW sensor or a one-port SAW sensor. The calibration sensor can be disposed on a printed circuit board of the implantable intraocular pressure sensor, such as any IOP sensor housings herein. The intraocular pressure sensor can include a piezoelectric sensor. The implantable housing can further comprise a rechargeable battery. The implantable pressure sensing device can further comprise at least one antenna adapted to provide at least one or data and power transfer. The implantable pressure sensing device can further comprise an external device that has stored in or more memory devices thereon any of the computer executable methods herein, wherein the external device and the implantable sensing device are adapted to wirelessly communicate to facilitate at least one of data transfer and power recharging. The implantable pressure sensing device can further include one or more storage devices that have stored thereon any of the computer executable methods herein related to calibration. The implantable pressure sensing device can further comprise a biocompatible coating disposed on at least a portion of the housing.

One aspect of this disclosure is a computer executable method stored on a storage device, the method adapted to be performed using a processor. The method can include receiving as input pressure information that is indicative of an output from an intraocular pressure sensor disposed in a housing of an implanted intraocular pressure sensing device, receiving as input calibration information that is indicative of an output from a calibration sensor disposed in the housing of the implanted intraocular pressure sensing device, using the calibration information to determine a correlation between the calibration information and an amount of fibrotic growth on the housing of the implanted intraocular pressure sensing device, and using the determined correlation and the pressure information to determine a corrected or modified intraocular pressure that corrects for fibrotic growth on the housing. The method may be performed using a system such as that shown in FIG. 26. The method can further comprise outputting the corrected intraocular pressure to a device, such as an external personal device, such as a smartphone.

Determining a correlation between the calibration information and an amount of fibrotic growth can include creating a mathematical relationship between the calibration information and the amount of fibrotic growth and is indicative of a calibration curve for the calibration information and the amount of fibrotic growth. Using the determined correlation and the pressure information to determine a corrected intraocular pressure that corrects for fibrotic growth on the housing can comprise using the mathematical relationship to determine the corrected intraocular pressure. Using the determined correlation and the pressure information to determine a corrected intraocular pressure that corrects for fibrotic growth on the housing can comprise applying a correction factor to the pressure information that accounts for the amount of fibrotic growth.

One aspect of the disclosure is a method of calibrating an implantable intraocular pressure sensing device, the method stored on a memory device. The method can include providing an implantable intraocular pressure sensing device that includes an intraocular pressure sensor and a calibration sensor that is adapted to create an output signal that is used to calibrate an output signal from the intraocular pressure sensor. The method can also include simulating fibrotic growth over at least a portion of the housing. After simulating fibrotic growth, an amount of simulated fibrotic growth can then be characterized. A pressure sensor output can then be obtained from the intraocular pressure sensor. A calibration sensor output (e.g. electrical signal) can be obtained from the calibration sensor. A correlation between the amount of simulated fibrotic tissue (indicative based on the calibration sensor output) and a corrected intraocular pressure can then be created. Creating a correlation between the amount of simulated fibrotic tissue and a corrected intraocular pressure can include creating a mathematical relationship between the calibration sensor output and an amount of simulated fibrotic tissue. Creating a correlation between the amount of simulated fibrotic tissue and a corrected intraocular pressure can comprise, based on the amount of simulated fibrotic tissue, creating a mathematical relationship between the pressure sensor output and the corrected intraocular pressure. Establishing this relationship can then be used, such as by executable methods herein, to create an accurate intraocular pressure measurement that takes into account an amount of fibrotic tissue growth on the implant.

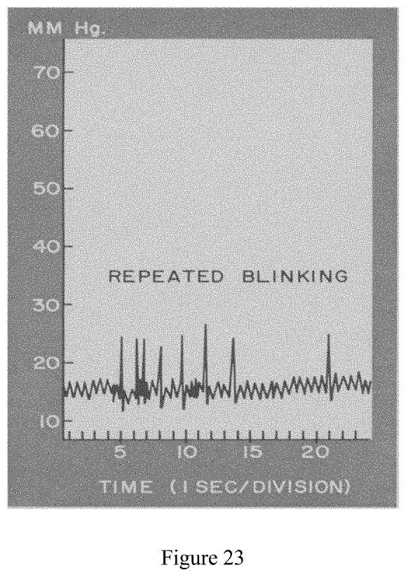

Another aspect of this disclosure is an alternative method of calibration used to normalize sensor sensitivity and response to a specific change in pressure. These alternative methods may also be used with methods and systems herein that accommodate for tissue growth on the implant. In this aspect, the calibration methods includes signal processing from the pressure sensor, and utilize fluctuations in the pressure of the aqueous humor due to natural blinks. IOP is known to fluctuate due to blinking or closure of eyelids, eye movements, head movements, and posture (lying down vs. standing), for example, as shown in the reference number fifteen referenced above. Among all these sources of high frequency IOP fluctuations, this disclosure includes using natural blink-induced IOP fluctuation as a reference. In these methods, the raw signal from the pressure sensor can either be processed by the logic circuit of the implant, or the raw signal can be exported to an external device (an “EID”) and processed there. FIG. 23 shows IOP data obtained on a human subject, with large variations due to blinking illustrated as the spikes in pressure. Variation of intraocular pressure from blinking depends on the individual patient and depends on the biomechanical properties of the sclera and blink force that is applied by the individual. The magnitude of this variation (typically, 8-12 mm Hg for humans and 5-8 mm Hg for non-human primates) is quite variable and needs to be calculated over a substantial number of blinks. The raw IOP data calculated in a signal processor in the implant or the EID (external device) can be smoothed in order to reduce noise, and then analyzed in the frequency domain in order to retain IOP fluctuations in the, for example, 100-500 msec range, rejecting IOP variations in the faster as well as slower time domains. The blink induced variation measured by the sensor can be normalized to a tonometer derived value obtained by a caregiver and entered into the EID (external device) as a calibration constant. This calibration constant remains independent of a gradual loss of sensor sensitivity due to, for example, accumulation of fibrous tissue (described above), and can therefore be applied to compensate for the change in sensor sensitivity. The calibration constant can be re-measured, for example once every six months or when the patient undergoes routine eye examination, since the magnitude of change in IOP caused by natural blinks may change if the patient develops any systemic eye disorder, especially ocular surface disorders.

One aspect of this disclosure is a method of creating a personalized correlation between blinking and intraocular pressure changes. The method can include, for a patient that has been implanted with an intraocular pressure sensing device comprising an intraocular pressure sensor, calculating measured intraocular pressure over a period of time based on an output from the intraocular pressure sensor, determining a blink-induced variation in intraocular pressure for the patient; and storing the blink-induced variation in IOP for the patient in a storage device. Storing the variation can comprise storing the variation in at least one of an external device and the implantable IOP sensing device. The method can also include determining an intraocular pressure reading for the patient that takes into account the blink-induced variation in IOP. The method can further include, after the storing step, performing the determining step again, such as at a next physician visit. Performing the determining step again can occur at least one month after the first determining step, and optionally up to six months after the first determining step.

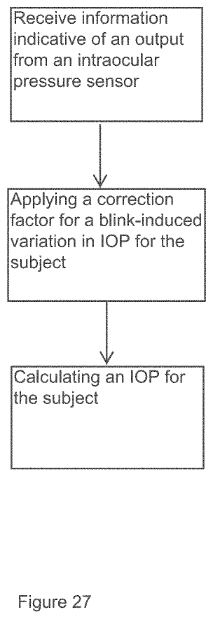

One aspect of the disclosure is a computer executable method stored on a user device. The method can include receiving as input information that is indicative of an output from an intraocular pressure sensor, and calculating an intraocular pressure sensor for a patient, while factoring in a personalized blink-induced variation of the patient. The method can be stored on an external device. FIG. 27 illustrates the general method. The method need not necessarily include the last step, and can be considered to only include one or both of the first two steps as shown in FIG. 27.

The disclosure herein related to calibration devices, systems and methods are understood to be practical applications of concepts that are specifically integrated into and used with intraocular pressure sensors. They are not merely mental processes, mathematical concepts, or methods of organizing human activity. Additionally, the calibration devices, systems and methods herein are significant improvements to technology. For example, the calibration sensors herein may be used to take into account a tissue growth on the implant, and can be used to arrive at an accurate pressure sensed from the IOP sensor. Additionally, the blinking calibration concepts herein are an improvement in technology in that they provide the implantable device with the ability to take into account an individual subject's blinking and its effect on IOP, which provides for more accurate IOP sensor readings.

In some embodiments the system includes one or more external interrogation devices (“EID”s) that are disposed outside of the eye and can be adapted to communicate (preferably wirelessly) directly or indirectly with the implant. The EID can be used to recharge a battery disposed in the implant, receive intraocular pressure data from the implant and/or reprogram the firmware embedded in an ASIC of the implant, when required. Communication between the implant and the EID follows a protocol, and example of which is shown in FIG. 16. This protocol involves encrypted data exchange, the encryption being compliant with all applicable Governmental regulations controlling confidentiality of medical information. Such a communication protocol also includes a handshake between the EID and the implant, the EID being the Master and implant being the Slave in this protocol. The exemplary protocol in FIG. 16 includes the following steps: 1) I am ready to transmit power and receive data; 2) I am ready to receive power, receive data, and I have data to transmit; 3) Transmission of data for initialization (code, time stamp, resonance frequency); 4) Data transmission (always recharging first step, when completed, data transmission (second step), when completed data transmission from External Unit to Implant (third step)); 5) Data transmission complete; recharging can begin in 2 seconds; 6) Wireless power transmission; 7) Threshold voltage reached, stop power transmission; 8) I am ready to receive data transmission (data for LUTs; reprogramming of firmware); 9) I have data/no data to transmit; 10) Data transmission, if step 9 gives code for data to transmit.

The one or more EIDs can receive information from the implant, such as pressure data (raw or processed) or other data indicative of pressure. The EIDs can also transmit information to the implant, such as instructions for programming or reprogramming some operational functionality of the implant (sensing software in the implant). One or more EIDs can also communicate with other EIDs, or external databases. An EID can also transfer power to the implant.

The systems herein can also include one or more software and/or firmware applications to collect, compile, and/or store individual sensor data (e.g., sensor measurements) for diagnostic or treatment evaluation support by the medical personnel (e.g., ophthalmologist). The software and/or firmware may exist on one or more EIDs, or in some instances may be disposed on or more implantable devices. The systems herein can also include one or more software applications to collect and/or compile multiple sensors data as a basis for medical data analysis, allowing support for, e.g., predictive medicine.

Management of data can include processing of raw signals to, e.g., filter noise and enhance signal to noise ratio, application of algorithms that recognize and select a true pressure data from spurious signals, further processing of data to, e.g., recognize and document 1 hour to 30 day trends in pressure, and reprogramming of the ASIC and device firmware in response to specific data trends or command by caregiver.

Theoretically, a truly continuous monitoring of IOP requires continuous monitoring of IOP at a frequency exceeding the most rapid spike in IOP recorded (approx. 30 Hz). In reality, the data generated by such a sensor will be of such a magnitude that it will be difficult to manage even with frequent downloading of data, and will also require a large battery in order to manage the daily power consumption of such a device. In some embodiments an optimum amount of pressure data is therefore collected per day, based on patient needs, needs of treatment, upper limit of power available, and size of the memory units in the device.

In some embodiments the resolution and accuracy of IOP data range from 0.2 mmHg to 1.0 mmHg and form 0.5 mmHg to 2 mmHg, respectively. In some embodiments the frequency of data acquisition is minimum 2/day to maximum 1/15 min. In some embodiments the frequency of recharge is less frequently than 1/day. In some embodiments the frequency of data transmission to a caregiver can be once a day or more. In some embodiments wireless recharging and data exchange is performed using inductive coupling or electro-magnetic coupling among magnetic and/or electric antennas respectively, uses a body safe frequency and intensity, and with minimum attenuation by human tissue. The implants should have a 10 years life of battery, and have hermetically sealed package.

The sensed data and/or data indicative of the sensed data can be stored in one or more proprietary databases. In some embodiments all of the database information must be reviewed by a physician before being included in the database. In these embodiments the patients do not have access to the database. One or more databases can store time histories of sensed pressure measurements, or time histories of data indicative of sensed pressure.

After implantation, the implant sensor senses pressure. Pressure can be sensed continuously (sensed during the entire time the implant is positioned in the patient, without interruption), or non-continuously. The implant can optionally have a continuous sensing “mode,” in which the implant is adapted to sense continuously, but the implant can also be taken out of the continuous mode, when switched to a different mode (e.g., no sensing, or a non-continuous sensing mode). When sensed non-continuously, it can be sensed periodically, either at regular intervals or non-regular intervals (e.g., sensed in response to detected events that do not happen with any known regularity). Exemplary regular intervals include one or more times a minute (e.g., 1, 2, 5, 10, 20, or 30 times a minute), one or more times a day (e.g., once, twice, five, twenty-four, 48 or 96 times a day). When sensed non-continuously, there may be epochs of time during which there is continuous sensing for a limited period of time, such as 1 minute of sensing, and then 59 minutes without sensing. An example of substantially continuous sensing is, for example, 30 times a minute. In some embodiments the pressure is sensed 1 time/day, or less (e.g., 1 time every two days). In some embodiments the frequency of sensing is between continuously and 2 times/day.

In some embodiments the implant is adapted to sense pressure at a particular frequency, but stores in memory only a subset of the sensed pressures. Sensed data can be stored in, for example, a first in first out manner.

The required IOP measurement pressure range can be, in some embodiments, 1 mmHg around ambient pressure and up to an overpressure of approximately 50 mmHg above ambient pressure.

In some embodiments the implant and one or more EIDs can be adapted so that the one or more EIDs can reprogram one or more functions of the implant. For example, an implant's sensing frequency, event detection, sensed threshold value, etc., can be reprogrammed by the one or more EIDs. Reprogramming can occur in response to a change in the database lookup tables, for example. Reprogramming can also occur in response to data sensed from the particular patient.

Any of the implants herein can have an internal power source that can be recharged using an EID. In some embodiments charging is done via an inductive or electromagnetic coupling with emitted powers from the EID in the 10-30 mW range, such as 25 mW, or in the range of 1 W to 5 W, such as 3 W. In some embodiments the EID can transmit power and data to the implant. In some embodiments the length of the antenna in the implant is 30 mm or less, such as 25 mm or less, such as 15 mm or less, such as 10 mm or less, and a height of 3 mm or less, such as 2.0 mm or less, such as 1.5 mm or less.

Even if not specifically indicated herein, one or more techniques or methods described in this disclosure (Including those related to factoring in blinking-induced variations in IOP) may be implemented, at least in part, in hardware, software, firmware or any combination thereof. For example, various aspects of techniques or components herein may be implemented within one or more processors, including one or more microprocessors, digital signal processors (DSPs), application specific integrated circuits (ASICs), field programmable gate arrays (FPGAs), programmable logic circuitry, or the like, either alone or in any suitable combination. The term “processor” or “processing circuitry” may generally refer to any of the foregoing circuitry, alone or in combination with other circuitry, or any other equivalent circuitry.

Such hardware, software, or firmware may be implemented within the same device or within separate devices to support the various operations and functions described in this disclosure, in addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

When implemented in software, the functionality ascribed to systems, devices, techniques and methods described in this disclosure may be embodied as instructions on a computer-readable medium such as random access memory (RAM), read only memory (ROM), non-volatile RAM (NVRAM), electrically erasable programmable ROM (EEPROM), Flash memory, and the like. The instructions may be executed by a processor to support one or more aspects of the functionality described in this disclosure.

1-17. (canceled)

18. A method of creating a personalized correlation between blinking and intraocular pressure changes, the method comprising:
in a patient that has been implanted with an intraocular pressure sensing device comprising an intraocular pressure sensor, calculating measured intraocular pressure over a period of time based on an output from the intraocular pressure sensor;
calculating a blink-induced variation in intraocular pressure for the patient, the blink induced variation caused by blinking; and
storing the blink-induced variation in intraocular pressure for the patient in a storage device.

19. The method of claim 18, wherein storing the variation comprises storing the variation in at least one of an external device or the intraocular pressure sensing device.

20. The method of claim 18, further comprising determining an intraocular pressure reading for the patient that takes into account the blink-induced variation in intraocular pressure.

21. The method of claim 18, further comprising, after the storing step, performing the determining step again.

22. The method of claim 21, wherein performing the determining step again occurs at least one month after the first determining step.

23. A computer executable method, the method comprising:
receiving as input information that is indicative of an output from an intraocular pressure sensor; and
calculating an intraocular pressure sensor for a patient, while factoring in a personalized blink-induced variation in intraocular pressure of the patient.

24. The method of claim 23, the method stored on an external device.

25. (canceled)
